(19) United States
(12) Patent Application Publication
(10) Pub. No.: US 2005/0101875 A1
(43) Pub. Date: May 12, 2005

(54) Non-invasive body composition monitor, system and method

(75) Inventors: Herbert J. Semler, Portland, OR (US); Philip D. Benz, Portland, OR (US)

Correspondence Address: IDEA ADVOCATES LAW GROUP LLC, 322 NW 5TH AVE #310, PORTLAND, OR, 97209, US

(73) Assignee: Right Corporation

(21) Appl. No.: 11/017,455

(22) Filed: Dec. 20, 2004

Related U.S. Application Data
(63) Continuation-in-part of application No. 09/971,507, filed on Oct. 4, 2001, now abandoned.

(51) Int. Cl. A61B 5/04; A61B 5/05

(52) U.S. Cl. 600/509; 600/547

(57) ABSTRACT

The invented non-invasive vital signs monitor is in a flexible, nominally flat planar form having integral gel electrodes, a sticky-back rear surface, an internal flex circuit capable of sensing, recording and playing out several minutes of the most recently acquired ECG waveform data and a front surface that includes an outplay port. The invented non-invasive body composition ‘risk’ monitor includes a measurement device for monitoring one or more variables including body fluid mass, dehydration, respiratory rate, blood pressure, bio-impedance, cardiography such as cardiac output, and body conformation parameters. The risk monitor may be provided in a lightweight carrying case into which the vital signs monitor plugs. Thus the two monitors may be independent or they may be integrated into one portable, non-invasive device that can convey important patient data to/from a remote patient medical data center via wireless telemetry for oversight, treatment and possible intervention by a physician.

This present invention is a continuation-in-part of prior application Ser. No. 09/971,507, filed Oct. 4, 2001, entitled DISPOSABLE VITAL SIGNS MONITOR, the disclosure of which is incorporated herein by reference.

### BACKGROUND OF THE INVENTION

The present invention relates generally to vital signs monitors whereby a patient's electrocardiograph (ECG), for example, is sensed and graphically recorded, e.g. as waveform data. More particularly, it concerns a thin flat, flexible monitor having integral electrodes that is extremely lightweight and may be adhered to the patient's chest during a recording session and that may be removed for local or remote outplay, as by mailing it to a physician's or diagnostician's lab for playout, diagnostic and/or archival purposes and ultimate disposal. The invented vital signs monitor lends itself to other continuous graphic waveform e.g. electroencephalograph (EEG) or pulse oximetry, or static, e.g. pulse-rate, blood pressure, glucose level, blood-oxygen level, vital signs monitoring, as well as telemetric control as for delivering pacer or defibrillation pulses to the monitored patient. The invented body composition or risk monitor lends itself to measurement and annunciation, recording and/or telemetry of data relevant to one or more of a patient's non-homeostatic body composition risk indicators, or indicia, including bio-impedance, water mass, respiratory rate, cardiography, e.g. cardiac output, height, weight, waist dimension and incline and standing/sitting position.

Some cardiac monitors having integral electrodes have been worn around the wrist, as described in U.S. Pat. No. 5,289,824 entitled WRIST-WORN ECG MONITOR, which issued Mar. 1, 1994. The high functional density of such cardiac monitors, and the provision therein of trans-telephonic communication of ECG waveform data to a remote physician site, render such monitors extremely useful in our increasingly busy and mobile society. More recent advances have rendered such high functionality and lightweight portability in the form of a credit card-shaped and -sized monitor such as the known HEARTCARD™ monitor. Such a product requires manual placement and slight pressure by the user on the monitor against the chest with the integral dry electrodes in contact with the skin and the manual depression of a record button. Such a product also requires the placement of a telephone call to a physician's office and the careful playing out of recorded, digitized, frequency-shift keyed (FSK) ECG waveform data via a telephone's mouthpiece. The HEARTCARD™ monitor is intended for long-term use, and thus is enclosed in a durable rigid housing, is provided with long-life batteries, and is supplied with a carrying case.

Other vital signs monitoring traditionally have included blood pressure, respiratory rate and body temperature, and a variety of methods for monitoring the same are known in the prior art. One emerging vital sign of vital importance to human health is bio-impedance, as it may be used as an indicator of cardiac output and fluid pressure drops, the latter being an earlier shock predictor than is a drop in blood pressure. It is reported that approximately half of the United States' population is overweight, and some reports suggest that 40-60% of the population is clinically obese. Morbid obesity, defined generally as persons weighing over approximately 300 pounds, is also on the rise: by some very recent reports, it has quadrupled since the 1980's. Obesity, which is preventable, can lead to Type I diabetes, cardiac arrest, cancer and/or even death. Indeed, statistics show that for a male with a waist circumference over forty inches or a female with a waist circumference over thirty-five inches, the risk of stroke or Type I diabetes is three to four times that of a person of more modest waist size. The cost of treating cardiac disorders resulting from obesity approaches $100 B annually just in the United States, and it is believed by many that, next only to tobacco smoking, obesity is the second greatest preventable killer.

Ironically, obesity worldwide now rivals hunger as a health hazard. It has the potential of overtaking smoking as the greatest preventable killer.

Ironically, poverty is responsible for most overweight and obesity. This is because cheap prepared food is fat- and carbohydrate-rich.

Body mass index (BMI) is a widely accepted measure of body mass, since it takes into account both weight and height, in accordance with a well-known formula. Generally, a BMI over twenty-five or thirty is considered a health risk. Morbid obesity is indicated with a BMI over fifty. Personal, so-called ‘bathroom’ scales often provide a measure of BMI, but the user must manually enter his or her height for the calculation to be accurate. Moreover, BMI fails to take into account other indicia of body mass that may implicate health. For example, and in accordance with the invention, the lateral slope of a person's belly can be an indicator of obesity, as can high blood pressure, low cardiac output or increases in bio-impedance.

Anorexia nervosa and bulimia also are on the rise as serious problems, as is human immuno-virus (HIV) or AIDS. These low body fluid mass conditions are preventable by intervention, medication and/or counseling. Nevertheless, detecting the conditions heretofore is not easy. This is because the conditions' characteristic behaviors often are subtle and most often proactively hidden by the victim. Moreover, like obesity, the early indicators of anorexia and bulimia are slight weight loss and patients that hope to hide their conditions may also be willing to lie to themselves and others about even the slightest recent weight losses or gains. Like victims of anorexia nervosa and bulimia, AIDS patients, unfortunately, sometimes simply give in to their disease and let it run its wasting course. Cardiac cachexia, another such wasting disease, also claims the lives of many people.

Obesity prevention preferably involves a combination of diet, exercise and monitoring. Anorexia and bulimia treatments involve a combination of counseling and monitoring. The importance of monitoring cannot be overstated. It provides essential feedback to a person at risk, whether positive or negative. Monitoring and reporting in real time is even more valuable, as it can immediately influence risky behavior or immediately reward measurable indicia of moderation of caloric intake or regimentation of cardiac output.

Several recent articles have been published regarding electrical bio-impedance measurements as they relate to various human subjects. These articles listed below may be referred to herein by their ordinal number, e.g. the Lukaski article may be referred to very simply as [3].

[1] Transthoracic Electrical Bioimpedance R-Wave Triggered Ensemble Averaging, anonymous article from Sorba Medical Systems, publication date unknwn, and related webpages describing non-invasive impedance cardiography.

[2] A Simple Way to Measure Intra and Extra Cellular Fluid/Bioimpedance Spectroscopy (BIS) Technology, anonymous, publication date unknown.

[3] H. Lukaski, Requirements for Clinical Use of Bioelectrical Impedance Analysis (BIA), Annals New York Academy of Sciences, pp. 72-76, publication date unknown.

[4] Casas et al., Ischemia, Annals New York Academy of Sciences, pp. 54-55, publication date unknown.

[5] Gheorghiu, et al., Impedance Spectra, Annals New York Academy of Sciences, pp. 68-69, publication date unknown.

[6] K. Ellis, R. Shypailo and W. Wong, Measurement of body water by multifrequency bioelectrical impedance spectroscopy in a multiethnic pediatric population, Am J Clin Nutr, pp. 847-853, 1999.

[7] A. Lackermeier, E. McAdams, G. Moss and A. Woolfson, In Vivo ac Impedance Spectroscopy of Human Skin/Theory and Problems in Monitoring of Passive Percutaneous Drug Delivery, Annals of New York Academy of Sciences, pp. 197-213, publication date unknown.

[8] J. Strobeck, M. Silver, Impedance Cardiography: Noninvasive Measurement of Cardiac Stroke Volume and Thoracic Fluid Content, Congestive Heart Failure, Volume 6, Number 2, pp. 3-6, March/April 2000 Reprinting.

[9] A. De Maria, A. Raisinghani, Comparative Overview of Cardiac Output Measurement Methods: Has Impedance Cardiography Come of Age?, Congestive Heart Failure, Volume 6, Number 2, pp. 7-18, March/April 2000 Reprinting.

[10] B. Greenberg, D. Hermann, M. Pranulis, L. Lazio, D. Cloutier, Reproducibility of Impedance Cardiography Hemodynamic Measures in Clinically Stable Heart Failure Patients, Congestive Heart Failure, Volume 6, Number 2, pp. 19-31, March/April 2000 Reprinting.

[11] J. Seibert, J. Wtorek and J. Rogowski, Stroke Volume Variability—Cardiovascular Response to Orthostatic Maneuver in Patients with Coronary Artery Diseases, Annals New York Academy of Sciences, pp. 182-96, publication date unknown.

Only one of the above articles remotely suggests body-worn bio-impedance monitors, and it teaches away from such devices. For example, [7] contains a section entitled BODY-WORN DEVICE but contains no teachings but the importance of electrode placement and the difficulty of incorporating impedance measurements into a body-worn device, for any purpose. Of course, the purpose of the work described in the article is transdermal drug delivery. There is no suggestion in [7] of non-invasive skin bio-impedance monitoring for overall body mass indicia, fluid body mass indicia or other non-homeostatic body composition assessment.

Fluid body mass is an important indicium of non-homeostatic body composition. It is believed that fluid body mass changes may be understood to indicate overweight conditions as well as underweight conditions. And it is believed that body fluid balance is an important and high-quality indicator of overall human health. As such, it is believed that body fluid mass monitoring and oversight can detect and can lead to treatment of, or perhaps even prevention of, obesity, anorexia nervosa and bulimia, HIV and cardiac cachexia, all of which are on the rise. All such abnormal conditions, whether absolute or relative, that are outside defined norms, i.e. are non-homeostatic, are candidate indicia for monitoring, oversight and intervention. Preferably, such body fluid mass monitoring is via bio-impedance, although, within the spirit and scope of the invention, any monitoring technique may be used.

Bio-impedance monitoring of humans has seen only limited use in medical diagnostics. This is because its early promise was derailed by success in alternative diagnostic techniques including magnetic resonance imaging, ultrasonic imaging and other non-invasive body scanning techniques. Nevertheless, bio-impedance monitoring is believed to represent an inexpensive, non-invasive technique for monitoring body fluid mass and fat content. As such, non-invasive monitoring and oversight can be achieved by the marriage of skin-electrode-based bio-impedance monitoring, body composition derivation, trend analysis and significant event or trend data conveyance via a common wired or wireless conveyance to a remote clinical site for physician oversight, treatment, medication and intervention.

### SUMMARY OF THE INVENTION

Briefly, the invented cardiac monitor is in a flexible, nominally flat planar form having integral gel electrodes, a sticky-back rear surface, an internal flex circuit capable of sensing, recording and playing out several minutes of the most recently acquired ECG waveform data and a front surface that includes an outplay port preferably having one or more snap connectors compatible with a lead harness from an n-lead recorder. The monitor has a relatively short battery life, as it is intended for limited-term use. After the patient has completed a recording session, the monitor may be simply sent in the mail to the prescribing physician for diagnostic and archival purposes. The physician or technician may play out the recorded ECG waveform data by activating an outplay mode of operation, and the patient's cardiography may be studied. The tiny, inexpensive monitor may then be disposed of, e.g., discarded or recycled. In a suggested alternative embodiment, the monitor further may be remotely controlled by telemetry to deliver pacer or defibrillation pulses to the patient.

Preferably, the monitor uses one or more zinc-air batteries the air inlet ports of which may be selectively configured, as by folding or otherwise manipulating the monitor's expanse, to either activate or deactivate particular recording or outplay modes of operation of the monitor. Thus, recording may be accomplished by simply opening the monitor, which activates the zinc-air batteries, and pasting the monitor on the patient's chest. When a recording session is complete, e.g. when a cardiac event has been detected or upon the initiative of the patient who may have sensed such an event, the monitor may be folded again thus deactivating the recorder by removing battery power therefrom. At the physician site, the opening again of the monitor may automatically activate an outplay mode of operation in which a connected n-lead recorder presents a strip chart recording of the patient's cardiography.

The circuitry within the flex circuit inner layer of the monitor's expanse may preferably be implemented by very large scale integration (VLSI) techniques by use of a custom integrated circuit (IC) that performs any necessary sensing, recording and outplay functions. The circuitry may be digital, and may include an analogue-to-digital (A/D) converter, a microprocessor with associated memory and a digital-to-analogue (D/A) converter. Alternatively, the circuitry may take the form of a direct analogue storage device having a differential amplifier front-end for sensing the amplitude of the analogue ECG input and having constant gain between input and output, the latter of which is coupled operatively with the outplay port Thus, outplay may be analogue or digital in form, and may be infrared (IR), audio (trans-telephonic), or electrical, e.g. an RS-232 serial input/output (110) port compatible with a connected personal computer (PC) or a lead-set compatible with an n-lead, e.g. a 12-lead, strip chart recorder. Other suitable recording and outplay means may be used such as a printer, tape, disk, CD-ROM, TV, VCR LCD, etc.

In accordance with another embodiment of the invention, non-homeostatic body composition monitoring method and apparatus are disclosed. A preferably portable, so-called ‘risk’ monitor measures one or more of the user's bio-impedance, cardiography including cardiac output, blood pressure, respiratory rate, body mass, water mass, dehydration, body fat and body conformation and position indicators such as height, waist diameter and/or circumference, lateral slope or incline and standing/sitting position. Preferably, the monitor then derives from such measured indicia and from information that may be entered manually or telemetrically indicia of body mass or fat and obesity or anorexia or bulimia risk index, annunciating and/or recording and telemetering such indicia and/or risk index to the user in a form of real-time feedback. The risk monitor preferably includes a housing, or carrying case, and is equipped with external electrodes extending from the housing in contact with the user's skin. A supplemental vital signs monitor can take the form of a flexible adhesive expanse, or so-called ‘patch’ having integral electrodes for cardiographic monitoring and relaying to the risk monitor. The risk monitor is capable of conveying recorded data to a remote site for oversight, treatment and possible intervention by a physician.

The preferred method of the invention involves equipping an at-risk patient with such a risk monitor, attaching electrodes to the patient's skin where appropriate for monitoring a desired vital and/or non-homeostatic body composition sign, and remotely conveying raw or calculated instantiation or trend data for oversight and/or treatment and/or medication and/or intervention purposes.

These and additional objects and advantages of the present invention will be more readily understood after consideration of the drawings and the detailed description of the preferred embodiment which follows.

### BRIEF DESCRIPTION OF THE DRAWINGS

FIG. 1 is a lateral, cross-sectional view of the cardiac monitor adhered to a cardiac patient's chest, showing some of the detail of its interior construction.

FIG. 2 is a schematic circuit diagram of the cardiac monitor made in accordance with a preferred embodiment of the invention.

FIG. 3 is a schematic circuit diagram of the cardiac monitor made in accordance with an alternative embodiment of the invention.

FIG. 4 is an isometric view of the monitor in a flat configuration in which it is useful for recording, and illustrates the laminar structure of the monitor of its preferred embodiment.

FIG. 5 is an isometric view of the monitor in a folded configuration that, in accordance with one aspect of the invention, protects its integral electrodes, powers-down its circuitry, saves its battery and readies it for a recording or outplay session.

FIG. 6 is an enlarged cross-sectional edge view of the invented apparatus taken generally along the lines 6-6 in FIG. 4.

FIG. 7 is an enlarged, fragmentary cross-sectional view of the apparatus taken generally along the lines 7-7 in FIG. 4.

FIGS. 8A and 8B are isometric views of the vital signs and body composition monitors proximate a patient and connected thereto in accordance with two embodiments of the invention by which body composition measurements such as body fluid mass and optionally other vital signs are measured, recorded and conveyed to a remote site.

FIG. 9 is a schematic block diagram of the body composition monitor made in accordance with yet another embodiment of the invention by which skin bio-impedance is measured using electrodes attached to the patient.

FIGS. 10A and 10B are simplified schematic block diagrams of the body composition monitor made in accordance with alternative embodiments of the invention by which a bio-impedance element is operatively coupled in the alternative with an ‘on-board’ or ‘out-board’ communication means.

FIGS. 11A, 11B and 11C are flowcharts of the invented method in accordance with alternative aspects of the invention.

### DETAILED DESCRIPTION OF THE PREFERRED EMBODIMENTS

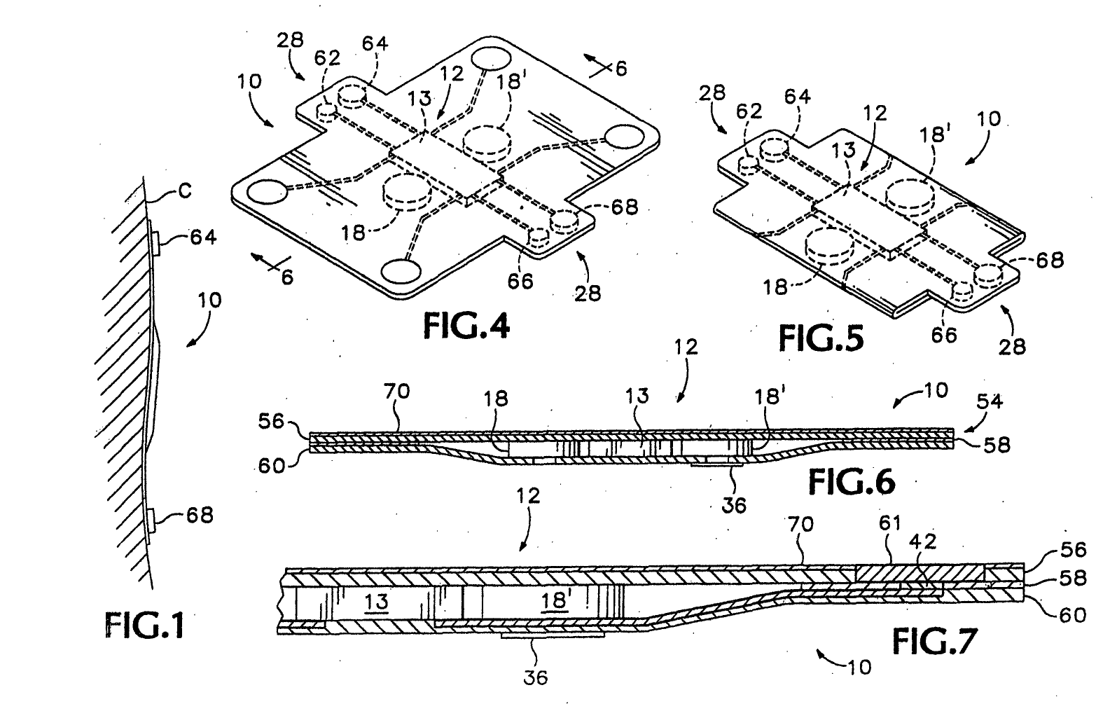

Referring first to FIG. 1, the invented disposable vital signal, e.g. cardiac, monitor is indicated generally at 10 adhered to the chest C of a medical patient. It will be appreciated that, because monitor 10 is integral, self-contained and adherent, the patient is free to move about performing everyday tasks without concern for lead-sets or external connections or manipulation of the monitor or any operator controls thereon. Because of its tiny size and weight, and because of its flexibility, the invented monitor resembles a medium-sized adhesive bandage, and thus provides for extremely convenient, affordable, comfortable and accurate vital signs monitoring and recording for children or men and women of all sizes and builds.

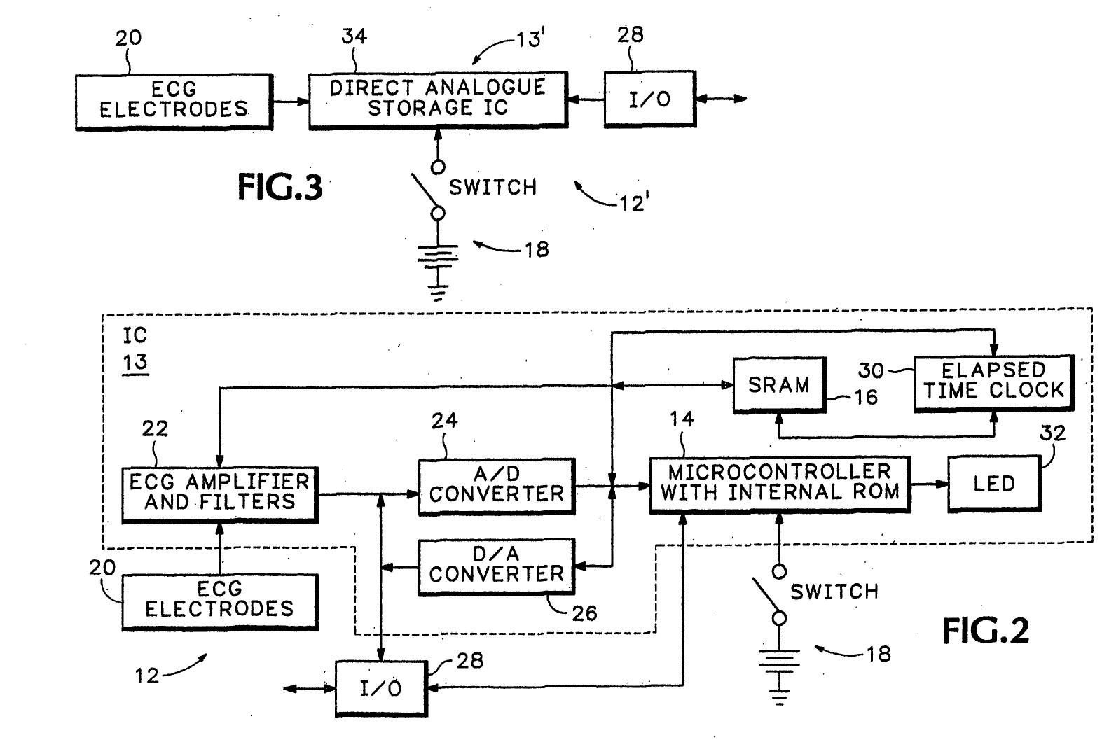

Monitor 10 will be understood to be capable easily and quickly of being removed by the patient at the end of a monitoring and recording session, thereby enabling waveform data recorded therein to be outplayed. Those skilled in the art will appreciate that outplaying may be via or to a local or remote presentation device such as a printer, tape, disk, CD-ROM, TV, VCR, LCD, etc. An outplay port is provided in monitor 10, as will be described in more detail by reference to FIGS. 2 and 3, in any of a variety of forms preferably including a set of snap connectors that are plug-compatible with the installed base of 12-lead strip-chart recorders found in diagnostic clinics around the world.

Those of skill in the art will appreciate that monitor 10 alternatively may utilize the world-wide web, or Internet, as a conduit or destination for the vital signs data stored therein. Thus, a so-called Bluetooth or other wireless, e.g. infrared or radio frequency (RF), interface port may be provided—compatible with the small size, thinness and flexibility of monitor 10—and vital signs data may be telecommunicated to nearby or remote sites via the Internet for playback, viewing, analysis, recording, archiving, etc. So-called Instant Messaging, a common feature of e-mail, could be used to post cardiograms to a receiving or diagnostic clinic or individual cardiologist situated anywhere in the world from a cardiac patient also situated anywhere in the world. Indeed, Instant Messaging could be used for duplex communications between a patient and a physician, however remote from one another, of vital signs data and other message content. For example, duplex communications can convey relatively static medical data about a patient such as height to the monitor and concurrently can convey dynamic vital signs and obesity risk data about the patient such as cardiac output or bio-impedance to the physician.

Thus, in accordance with the preferred embodiment of the invention and method for its use monitor 10 may be purchased over-the-counter by a medical patient and upon completion of a recording session may be delivered, as by mail or walk-in or drive-through, to a diagnostic clinic for outplay, oversight, diagnostics and archival recording. Because it is meant for limited-term use, and is extremely inexpensive to manufacture, after its recorded data is outplayed at the clinic, monitor 10 may be disposed of, e.g. discarded or recycled, much like a disposable flash camera. Of course, those of skill in the art will appreciate that, within the spirit and scope of the invention, monitor 10 instead may be reused, as by recharging or replacing one or more batteries, which it is appreciated typically might require some rebuilding of the novel laminar structure and thus may not be cost effective.

The invented vital signs monitor, then, may be seen most broadly to include a flexible generally planar expanse that includes a front surface and a rear surface including a region capable of being adhered to a patient's skin, with the rear surface bearing two or more, e.g. four, electrodes. Preferably, the monitor includes also an outplay port, as will be seen, that may take the form of a general-purpose input/output (I/O) port that is wired or wireless and that enables an interior flexible circuit sandwiched between the rear and front surfaces of the expanse to communicate either unidirectionally or bidirectionally with an external device such as a remote transmitter/receiver or processor or simple hardcopy device.

Those of skill in the art will appreciate that FIGS. 1, 4 and 5 show monitor 10 in a given size that may be suitable for adherence to the chest of a person of average size. Within the spirit and scope of the invention, disposable vital signs monitor 10 may assume a variety of sizes, e.g. adult (e.g. over eighteen years), youth (e.g. between 11 and eighteen) and child (e.g. under eleven) sizes, compatible with more individualized torsos. Such may be particularly beneficial for monitoring sudden infant death syndrome (SIDS) most likely to strike infants. Importantly, the thin, lightweight, flexible monitor imposes little or no burden or inconvenience even for a person having the most fragile frame or tiny body. Thus, SIDS among other anomalies or syndromes may be monitored, and lives may be saved, using the invented disposable vital signs monitor even in the case of a preemie of extremely low birth weight and size, and the same or other vital signs may be monitored even in the case of a weak and/or disabled elder.

High-risk athletes or non-athletes also are candidates for use of the invented vital signs monitor. Athletes could wear the monitor under their normal athletic attire during a sporting event, without adverse effect on their performance, but with the possibility of discovering and treating an anomaly. High-risk patients, for example, during the post-myocardial infarction (MI) or post-coronary angioplasty (PCTA) phases of their treatment may be equipped with the vital signs monitor to record and early detect or diagnose any anomalous vital signs that are monitored thereby during critical post-operative or post-treatment phases of their lives. Those of skill in the art also will appreciate that the invented vital signs monitor may be used on non-human patients. In other words, veterinarians might use the vital signs monitor on dogs, cats, horses or other animals in the delivery of veterinary health care.

Turning now to FIG. 2, a schematic diagram of the interior flexible circuit or circuitry of the preferred embodiment of the invention is shown at 12. It will be appreciated that circuitry 12 preferably is implemented in one or more integrated circuits or other integral components of extremely light weight, low profile and small footprint. Such may be one or more highly integrated circuits (IC), as is taught by the above-referenced patent disclosure. Those of skill in the art will appreciate that circuitry 12 may provide more or less functionality than is described herein in terms of a preferred embodiment of the invention, within the spirit and scope of the invention. For example, circuitry 12 may include pulse generation means that, via the same gel electrodes as those used for monitoring, deliver a series of low-wattage pacer pulses or a high-wattage defibrillation pulse to the patient's heart.

Referring now in more detail to FIG. 2, it may be seen that circuitry 12 preferably includes a micro-controller 14, or a microprocessor having internal read-only memory (ROM) suitably programmed; non-volatile, e.g. static, read-and-write memory (SRAM) 16 for variable and vital signs waveform data recording or storage; at least one battery 18 selectively operable to power and thus enable the circuit to perform its sensing, recording, producing and playing functions. Battery 18 preferably is of the air seal type, e.g. one or more zinc-air batteries of which only one is shown in FIG. 2, having an integral SWITCH for selectively applying power to the remainder of circuitry 12; plural electrodes such as the preferred gel-type ECG electrodes indicated generally at 20; signal-sensing circuitry such as ECG amplifiers and filters 22 operatively connected with electrodes 20; an analogue-to-digital converter (ADC) 24 that operatively couples the electrodes to the digital processor operatively coupled, in turn to the memory; a digital-to-analogue converter (DAC) operatively coupled with the digital processor and the memory and operatively coupled, in turn to an outplay port; and an input and/or output (I/O) or more simply an outplay port indicated generally at 28 for conveying sensed and recorded vital signs waveform data to a remote outplay or recording device for medical diagnostic purposes and, optionally, for receiving command or control data from a nearby preferably wireless transmitter for cardiac pacing or defibrillating purposes.

Those skilled in the art will appreciate that, by logical extension, disposable vital signs monitor 10 may be of the so-called Holter monitor-type characterized as providing multiple-lead cardiac monitoring. Such a monitor might use any suitable arrangement or number of leads both within the perimeter of the monitor's body, as illustrated in FIGS. 1, 4 and 5, or having external leads attached to thin, lightweight cables extending therefrom. In such an arrangement, the monitor itself yet might be disposable after, say, 24-48 hours worth of cardiac data are monitored and continuously recorded. Alternatively, a looping memory scheme may be used, as is known but as will be described briefly below, to selectively record only more pertinent, suspected event, data for much longer periods of time, say 1-2 months. Those of skill in the art will appreciate that the volume of data recordable in memory, whether continuously or selectively, increases step-wise periodically, as semiconductor memory densities increase and prices decrease.

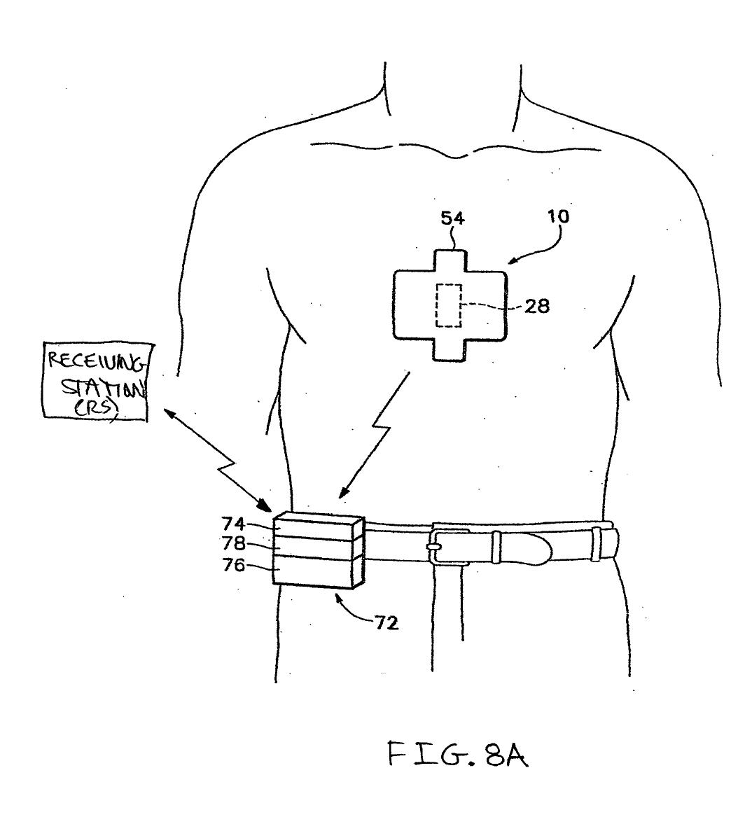

It will be appreciated that such circuitry 12 as described above readily may be integrated into one or more custom integrated circuits (ICs) that take up little space, whether in the plane of monitor 12 or normal thereto. Preferably, one IC 13 is used to reduce cost and flex circuit and interconnect complexity, as suggested by the simple configuration of monitor illustrated in FIG. 4, to be described below.

Those skilled in the art will appreciate that circuitry 12 also may include an elapsed time clock 30 for data-and-time stamping of recorded vital signs waveform data and one or more audio or visual annunciators such as beepers or light-emitting diodes (LEDs), e.g. LED 32, for indicating to the patient or clinician the status of monitor 10, i.e. whether it contains recorded vital signs waveform data that is ready for outplay.

Within the spirit and scope of the invention, circuitry 12 may provide other useful functions. For example, a scrolling or looping memory function may be provided by which SRAM 16 is partitioned into one or more looping buffers for the capture-store of a predetermined time duration of data, with the most recently sensed, i.e. the latest recorded, data always present therein and with the least recently sensed, i.e. the oldest recorded, data lost. In this way, circuitry 12 equipped to trigger on a detected cardiac anomaly may halt recording of data into the looping memory thereby to capture for outplay a cardiac data window that is pertinent to, because it is time proximate to, the triggering cardiac event. Numerous alternative or additional functions may be provided by circuitry 12, within the spirit and scope of the invention, as it is understood that functionality readily may be added by reprogramming or masking a state or logic controller such as microcontroller 14.

FIG. 3 schematically illustrates an alternative embodiment of the circuitry that may be used within monitor 10 to implement the basic sensing, recording and outplaying functions. Circuitry 12′ provides such functions in the form of an analogue signal recorder such as those used to customize greeting cards by permitting the sender to record a message which is outplayed automatically when the recipient opens the greeting card. Such analogue 10 memories, or direct analogue storage devices, such as that indicated at 34 (also designated 13′ to indicate that it is counterpart to digital IC 12 of FIG. 2) are inexpensive to manufacture, and have a recording capacity-because of the unique nature of vital signs waveform data-of recording at least approximately one minute of continuous ECG waveform data sensed by the electrodes, preferably at least approximately two minutes thereof and most preferably at least approximately four minutes thereof.

The differences between the human voice and vital signs graphic waveform data lead to this eight-fold recording capacity increase. The human voice may be reasonably well reproduced by digitizing it at a sampling rate of approximately 4000 Hertz (Hz), whereas accurate cardiac graphic waveform data need be sampled only at approximately 400-500 Hz in order to faithfully reproduce it for a clinician to diagnose the shortest duration arrhythmic, ischemic or other cardiac anomaly. Moreover, because of the analogue nature of the stored data, representing essentially in a single sample the amplitude of a patient's skin potential between two electrodes is possible with direct analogue storage, whereas eight binary bits typically are used to represent a digital representation of such amplitude. Thus, by lowering the sampling rate of such a device, its capacity to record vital signs graphic waveform data is greatly increased to a meaningful level.

Whether monitor 10 stores a digital or an analogue representation of the sensed vital signs waveform signal, it is preferably in accordance with the invention that at least approximately one minute of such sensed vital signs, e.g. ECG, signal be recorded within memory 18 or 18′. More preferably, at least approximately two minutes of such sensed vital signs signal is recorded, and most preferably approximately four minutes of capacity within memory 18, 18′ is provided, thereby rendering monitor 10 useful for multiple event or medium-term monitoring of patient vital signs. It will be appreciated that the useful capacity of memory 18 or 18′ may be effectively increased by the use of scrolling or looping memory and automatic trigger event-detection such that the greatest fraction of recorded vital signs signal is useful in representing the patient's vital signs for overview and analysis by a diagnostician.

Other modifications are required to such a direct analogue storage device to render it suitable for vital signs monitoring. First, the input amplifier section must be made differential so match the differential input from the electrodes, as may be readily accomplished by those of skill. Second, the gain of the device must be made substantially constant, or of substantially consistent unity gain, from such differential input to output. Such straightforwardly may be accomplished by simply disabling the automatic gain control (AGC) of the conventional direct analogue storage device.

Operatively connected to the differential input terminals of such analogue storage device 34 is an electrode pair, or ECG electrodes 20 made in accordance with the preferred embodiment of the invention, which electrodes of course carry a differential signal representing the patient's skin potential (typically a third and fourth electrode provide a common baseline for the differential pair). Operatively connected to the output buffer electronics of such analogue storage device 34 is bidirectional I/O, or unidirectional outplay, port 28 also made in accordance with the preferred embodiment of the invention, which outplay port of course may take any of the variety of forms described or illustrated herein. One or more identical batteries such as illustrated battery 18 may be used, connected to the analogue storage device preferably via a battery-integral SWITCH, as shown.

As indicated, it is preferable that a reserve battery (not shown in FIGS. 2 and 3 for the sake of clarity, but shown in FIGS. 4, 6 and 7 described below) be provided as back-up to primary battery 18 in both the preferred and alternative embodiments illustrated in FIGS. 2 and 3, in case the primary battery fails. Those of skill in the art will appreciate that the primary and reserve batteries may be connected in parallel so that whichever one has sufficient power and has its integral switch (air-powered) will supply the remainder of circuitry 12 or 12′ within monitor 10. Alternatively or additionally, and within the spirit and scope of the invention, one or more higher capacity batteries may be provided, thereby enabling pulse generation circuitry within monitor 10 to deliver relatively high-voltage pacer or defibrillation pulses to the patient.

FIG. 4 shows monitor 10 in a bottom isometric view in its flat configuration for medical patient waveform data recording, i.e. in what will be referred to herein as its second, deployed configuration. In its preferred embodiment, the laminar structure may be seen to take the form of a thin preferably rectangular, generally planar expanse that will be understood by its structure to be flexible. The thin rectangular expanse may be approximately credit card-shaped and sized, or approximately 6.0 cm×9.0 cm×0.4 cm (2.4″ ×3.6″ ×0.16″). Those of skill in the art will appreciate: that monitor 10 may take alternative shapes and sizes, within the spirit and scope of the invention. It will also be appreciated that, if made to be credit card-shaped and -sized, monitor 10 may have the additional feature of a ROM magnetic strip on one edge thereof that may be initially programmed to identify the patient to whom the monitor is provided and that may later be read by a suitable magnetic strip reader. Such a ‘smart’ card approach is within the spirit and scope of the invention.

Within a preferably central interior region of monitor 10 are one or more batteries such as primary and reserve zinc-air batteries 18, 18′ operatively interconnected preferably by an air-actuated switch integral therewith to circuitry 12 capable of sensing, recording and outplaying vital signs waveform data such as a patient's ECG waveform. It will be appreciated that primary battery 18 has its air inlet normally exposed on the front surface of the expanse of monitor 10 so that it is operative when monitor 10 in its second, deployed configuration is tightly adhered to the patient's chest as in FIG. 1. It may be seen that normally air inlet of reserve battery 18′ is covered by an air-impermeable sealing tab 36, as shown. This way, the battery is not normally in operation but may be easily rendered operative by the tab's removal.

Recent advances in battery technologies render far greater performance to disposable vital signs monitor 10. It is believed that a sheet battery is presently under development by the military that could be used to power the relatively low-power requirements of monitor 10 as described herein. Such a battery is made of a special laminar fabric which may be cut to size and which exhibits a sustained electrical potential thereacross capable of powering one or more electrical circuits. Such a recent advance might prove extremely suitable as a suitable alternative to the discrete one or more batteries illustrated herein, because of the similar characteristic flexibility of such sheet batteries and the disclosed monitor, leading to even thinner and more flexible disposable vital signs monitors. One such sheet battery, the Power Paper™ thin battery, is available from Power Paper Ltd., an Israeli corporation. It is contemplated that, within the spirit and scope of the present invention, some or all of the circuitry including the electrodes, the flex circuit, the memory and/or processor chip and the batteries may be integrated into a thin, laminar configuration.

In accordance with a preferred embodiment of the invention, four gel-type electrodes 38, 40, 42, 44 are provided in the four corners of the expanse on the bottom surface thereof for contact with the patient's chest. Preferably, such electrodes which are referred to collectively herein as electrodes 20 are connected with corresponding input terminals of circuitry 12 in accordance with one of the schematic diagrams of FIGS. 2 and 3, discussed above via a flex circuit conductor layer that also connects the batteries with the remaining circuitry. This flex circuit conductor layer is indicated somewhat schematically in FIG. 4 by dashed line pairs extending from circuitry 12 to batteries 18, 18′, to electrodes 20 and to outplay port 28 (this flex circuit is illustrated in more detail in FIG. 7).

It will be appreciated that, alternatively and yet within the spirit and scope of the invention, electrodes 38, 40, 42, 44 may be of another type of so-called wet electrodes, or even may be dry electrodes as are taught in the above-referenced patent disclosure. It will also be appreciated by those skilled in the art that the number, configuration and spacing of electrodes 20, within the spirit and scope of the invention, may vary depending upon the cardiac (in the case of ECG), cerebral (in the case of EEG) or other vector(s) to be monitored and recorded by monitor 10. It will also be appreciated that electrodes 20 of the gel type are suitable for use in pacer and defibrillation pulse transmission to the patient.

Shown in FIG. 4 as four snap connectors 46, 48, 50, 52 (indicated by dashed lines) and associated I/O routing flex circuitry (in pairs of dashed lines) is I/O or outplay port 28. It will be appreciated that snap connectors 46, 48, 50, 52 may be located anywhere in the flexible expanse of monitor 10 that does not interfere with its use in recording and outplaying sessions. The chosen position permits monitor 10 to be flatly bi-folded as shown in FIG. 5 to seal the air inlets of batteries 18, 18′, while not measurably increasing the overall profile of monitor 10. It will be appreciated that placement of connectors on the rear surface or edge surfaces of monitor 10 may be possible within the spirit and scope of the invention, without interfering with adherence by monitor 10 to the patient's chest or accurate sensing of vital signs thereat, depending upon their physical configuration.

It will also be appreciated that edge connectors may be used that are within the slight overall profile of monitor 10. For example, many so-called PCMCIA modem cards present a phono jack for telephone cord connection in the extremely thin edge regions thereof, and such might be used with a different type of I/O port envisioned by the invention. With wireless communication schemes such as IR or RF or audio (e.g. trans-telephonic), extremely low- or no-profile I/O ports alternatively may be provided. For example, IR may be-used to provide bidirectional wireless communication between the monitor and a nearby receiver, akin to the use of a wireless remote control on a television or a vehicle security system. All are within the spirit and scope of the invention. Alternatively, monitor 10 may be equipped with an internal modem as part of circuitry 12, thereby enabling direct telephone line connections for remote outplay. All such producing and playing of waveform data functions of circuitry within the expanse are contemplated and are within the spirit and scope of the invention.

Brief reference to FIG. 5 shows monitor 10 in what will be referred to herein as its first, stowed configuration in which the air inlet to battery 18 is substantially closed or covered by one folded expanse, thereby rendering battery 18 inoperable, via its integral SWITCH, to supply power to circuitry 12. In this stowed configuration, the monitor safely and confidently may be transported or stored, e.g. in a flat envelope, without decreasing battery life and without risking loss of any patient vital signs waveform data stored in its non-volatile memory. It will be appreciated that a paper backing sheet cut approximately to the rectangular shape and size of monitor 10 when flat might be placed on the adhesive-coated rear surface thereof when monitor 10 is not being used to record vital signs data thereby protecting electrodes 20 from wear or contamination and a patient's or clinician's hands from stickiness. Those of skill in the art will appreciate that manipulation of the monitor's expanse from the first, stowed configuration shown in FIG. 5 to the second, deployed configuration shown in FIG. 4 selectively operates the battery (e.g. by supplying its air inlet with air by unblocking it), thereby to power and thus enable the circuit to operate, e.g. for recording or outplay.

It will be appreciated that, in accordance with an alternative embodiment of the invention, monitor 10 need not be folded or configured specially for stowage. In such an alternative embodiment, the air inlet of battery 18 might be sealed by simply placing a sealing tab thereover, i.e. to save primary battery 18 when it is not needed just as reserve battery 18 is saved when it is not needed. Such a flat configuration of monitor 10 whether in operation or not lends itself to the ‘smart’ card magnetic encoding described above. Nevertheless, by the use of air seal batteries in a disposable vital signs monitor, no physical pushbutton switch or other operator control is required to operate monitor 10 in all of its intended functional roles. Thus, unnecessary cost, weight and complexity in monitor 10 are avoided.

As may be seen by reference to FIGS. 6 and 7, the generally planar expanse (designated 54 therein) may include three white foam electrically insulative layers 56, 58, 60 of the type that are used in gel electrodes such as the medical electrode foam available from 3M®. A bottom layer 56 preferably covered or coated with what may be an electrically conductive adhesive coating or layer 70 has formed therein four electrically conductive gel electrodes (only one 42 of which is visible and only in FIG. 7) typically formed using metal powders and gels as in the formation of gel electrodes. A middle layer 58 extends around the perimeter of monitor 10 and is adhesive, thus serving when the laminar structure is conventionally cured as by heating to seal the perimeter, or edge, of the monitor. A top layer 60 is the flex circuit layer that routes signals among the circuitry components such as the battery, the electrodes and the digital or analogue processor/memory IC 13 or 13′. A conductive run of the flex circuit layer, which electrically connects electrode 42 with circuit 13, is illustrative of such circuit layer in cross-sectional view.

The flex circuit laminate or substrate for the ICs may include either a so-called complete flex or a so-called rigid flex circuit board material in which, respectively, the entirety or only a region of the patterned circuit area (shown in FIG. 7 in cross section) is flexible. It will be appreciated that—due to the very large scale integration (VLSI) of IC 13 or 13′ and the few associated circuitry 12 components including batteries 18, 18′, electrodes 20 and I/O port 28—very few signals are required to be routed in the flex circuit layer. As a result, a single-level flex circuit layer, a part of which is shown in FIG. 7 in cross section, may be formed conventionally and with very low-resolution patterning, e.g. photo-lithographic copper powder deposition, for example, thereby further reducing the cost of monitor 10.

Circuitry 12 including IC 13 or 13′ and batteries 18, 18′ may be seen essentially to be sandwiched in the void between the bottom and top layers of the foam laminate of which electrodes 20 preferably are an integral part. Preferably, IC 13 or 13′ is of the surface mount technology (SMT) type, thus producing an extremely low profile, e.g. less than approximately 0.4 cm (0.16″), laminar structure even in the central circuitry-containing region of monitor 10. Alternatively, chip-on-board techniques may be used to mount circuits and to route signals among components including ICs, batteries, electrodes and I/O ports.

Preferably, the four or more electrodes are connected to the inputs of the differential amplifier of the sensing circuit via a corresponding number of metal posts, e.
g. metal post 61 electrically coupled with electrode 42, that extend outwardly from the gel electrodes and through the insulative inner layer, the posts being connected to flex circuit solder pads corresponding to such inputs, as shown. Such through connections from the inner to the outer laminar foam layer may of course be accomplished in any suitable manner, as via plated-through holes, or so-called vias, formed within a flexible, multi-layer chip-on-board circuit and interconnect configuration.

It will be appreciated that one or more outplay ports may be provided in monitor 10 to achieve a desired price-performance level and compatibility with local or remote outplay, data communication and recording equipment. Referring briefly to FIGS. 4 and 5, it may be seen that preferably one or more, e.g. four, snap connectors 62, 64, 66, 68 are provided extending from the front surface of monitor 10 for plug compatibility with 12-lead recorders. Additionally or alternatively within the spirit and scope of the invention, additional snap connectors, an RS-232 serial I/O port, an RF or IR receiver/transmitter port, a telephone jack and/or a speaker may be provided to render monitor 10 compatible with a wide variety of unidirectional or bidirectional communication, hard-copy and recording devices. It also will be appreciated that an LED or beeper may be provided that informs the patient that a recording has been made and/or that memory is full of vital sign data, so that the patient knows when to remove monitor 10 from the body and to locally outplay the data for diagnostic purposes or to surrender the monitor with its data contents intact for diagnosis at a remote site.

FIG. 5 shows monitor 10 in a second, folded configuration in which the air inlets of primary zinc-air battery 18 (not visible in FIG. 5—refer to FIG. 4) is substantially closed, thereby depriving the battery of air and the circuitry of power. The controller within monitor 10 in this configuration goes into a power-save mode of operation in which memory containing a recorded vital sign graphic waveform is preserved but very little power is consumed. It will be appreciated that when monitor 10 is received at the diagnostic clinic, it very simply may be unfolded to reenergize the battery and outplayed to a desired hard-copy or recorder device such as a 12-lead recorder via the snap connectors.

In the event that the primary, preferably air-seal, e.g. zinc-air, battery 18 is dead, when monitor 10 is received at the clinic, a backup battery 18′—having a normally affixed tab 36 over its air inlet—may be used to play out the recorded cardiac waveform data. This is accomplished very simply by uncovering the air inlet over the reserve zinc-air battery. The controller, which is ‘aware’ that it has recorded ECG waveform data in its memory, preferably automatically exits the battery-save mode and-a predetermined number of seconds after the clinician unfolds the monitor-outplays the waveform data stored therein.

Broadly speaking, then, the invented disposable vital signs monitor may be seen to represent a significant improvement in portable, self-contained medical patient vital signs monitoring and control wherein such a monitor includes a generally planar expanse including a front surface and a rear surface having integral electrodes and having between the front and rear surfaces circuitry capable of sensing a vital signs signal present on the electrodes, recording the sensed signal and outplaying the recorded signal to an external device. The improvement may be understood to involve, most importantly, rendering such an expanse flexible and conformable to the shape of a patient's body, thereby to greatly improve the sensitivity and accuracy of such monitoring. Preferably, as described and illustrated herein, the monitor is also rendered self-adherent to the patient's body, thereby obviating cumbersome handling by the otherwise ambulatory patient. Also as described and illustrated herein, the monitor preferably is rendered capable of being controlled by remote telemetry, as via the provided I/O port in the wireless ones of its disclosed embodiments.

Preferably, the vital signs that are within the monitoring capability of such an improved monitor include ECG, and the monitor includes integral gel electrodes, which have been found further to increase the sensitivity and accuracy of such ECG monitoring. Within the spirit and scope of the invention, however, EEG, pulse oximetry or other continuous, real-time medical patient waveform monitoring is contemplated. In the case where ECG is the vital sign being monitored, the monitor may be rendered capable of being controlled by remote telemetry, wherein it is rendered capable of pacing a cardiac patient being monitored thereby by remote control, as described above. Moreover, as taught herein, the monitor may be rendered capable of defibrillating such a cardiac patient whose ECG is being monitored thereby.

In those cases where the vital signs being monitored include ECG, and where the monitor is equipped with cardiac event-detection capability, the monitor preferably may be equipped with a looping memory for continuous recording and window captured-data outplaying of a buffer representing—at the time of outplay thereof—a sensed ECG waveform signal that is related in time to a detected cardiac event, Such a scrolling memory feature is described in detail above and in the above-referenced patent, and, by saving on memory capacity, minimizes the circuitry required to implement the required functionality in a tiny, thin, planar flexible expanse that—due to its low cost—may be readily disposed of or recycled after use.

FIGS. 1, 4 and 5 perhaps best illustrate use of invented disposable cardiac monitor 10. FIG. 1 shows monitor 10 in its deployed configuration, albeit a lateral, cross-sectional view thereof, i.e. flattened out and adhered via a preferably conductive adhesive coating or layer 70 (shown for the sake of clarity only in FIG. 7) to a cardiac patient's chest C; FIG. 4 shows monitor 10 in its same deployed configuration but in a helpful isometric view; and FIG. 5 shows monitor 10 in its stowed configuration (in an isometric view corresponding with that of FIG. 4), i.e. bi-folded and ready to insert into a mailing envelope to send to a diagnostic center. Importantly, with monitor 10 in its deployed configuration, primary zinc-air battery 18 is operable to power circuitry 12 that senses, records and outplays vital signs waveform data, and, with monitor 10 in its stowed configuration, zinc-air battery 18 is inoperable to power circuitry 12, thus greatly extending battery life and eliminating the need for pushbuttons or other patient or physician controls.

Those of skill in the art will appreciate that the invented flexible monitor also far better conforms to the patient's chest, which may be irregular or even scarred, and utilizes gel electrodes rather than dry skin electrodes, thus increasing the integrity of-cardiac waveform data sensed therethrough. Accordingly, diagnostic accuracy is improved, yet in an extremely inexpensive-to-manufacture, easy-to-use device. It also will be appreciated that the disposable vital signs monitor may find application in areas other than cardiac monitoring. For example, electroencephalograph (EEG) or pulse oximetry waveform monitoring are also possible, as well as more static medical patient vital signs monitoring such as pulse-rate, blood pressure, glucose level, blood-oxygen level, etc. Such may require a transducer of a different form to convert a patient's body characteristic signal into data suitable for recording and outplay, but any one or more lend themselves to the convenient, lightweight, inexpensive form of the invented disposable vital signs monitor.

In accordance with an alternative embodiment, a monitor that also is capable of acting as a pacer or defibrillator may be remotely controlled by a nearby transmitter to which its I/O port is programmed to respond. An ambulatory cardiac patient who is visibly experiencing tachycardia or other arrhythmia may be treated by a bystander equipped with such a portable, hand-held transmitter that may resemble, for example, a television remote control device. Valuable seconds, perhaps critical seconds, may be saved by such a remote pacer or defibrillator function provided by circuitry 12, as described above, using the proposed telemetry which requires only that I/O port 28 have bidirectional capability and that microcontroller 14 and associated circuitry provide pulse generation means, as is known.

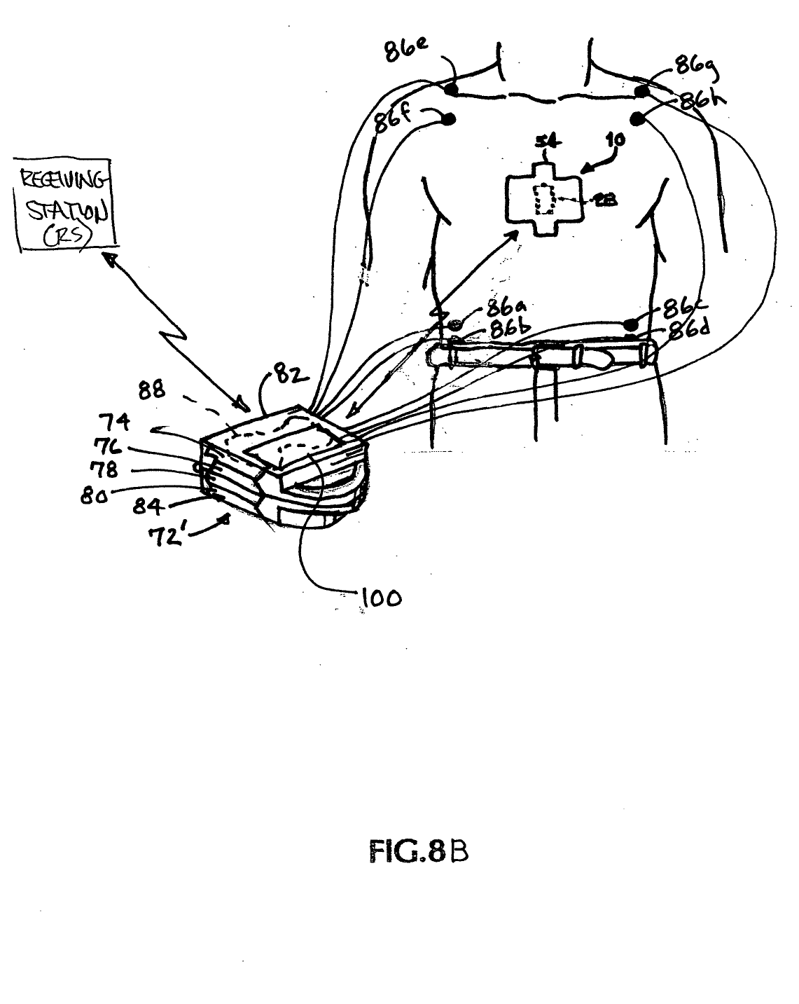

Alternative configurations for disposable vital signs monitor 10 are contemplated as being within the spirit and scope of the invention. For example, components of the monitor may be removed from the integral flexible expanse 54, which will be referred to hereinafter as a flexible housing, to a remote, preferably portable and belt- or body-worn device 72 having its own housing. FIG. 8A shows such a configuration in which, for example, auxiliary I/O ports 74, 76 and an auxiliary battery 78 are provided. Auxiliary I/O port 74 will be understood to support a wired or wireless, e.g. IR or RF, telecommunications and optional power interface to I/O port 28 (see FIG. 2). (Auxiliary I/O port 76 will be understood to support wired or wireless, e.g. IR or RF or audible, telecommunications with, for example, a conventional telephone handset or acoustic coupler (not shown in FIG. 8A). Alternatively, I/O port 76 may support wired or wireless telecommunications directly with a remote site's Receiving Station (RS), as indicated.) In this configuration, power may be provided or augmented via auxiliary battery 78 to the electronics within housing 54 (via a power cable or harness not shown), as well as to the auxiliary I/O ports 74, 76.

Thus, additional hardware of any suitable function may be provided in a convenient auxiliary device 72 operatively coupled with a patient chest-worn device 10. Indeed, auxiliary device 72 may include conventional cellular telephone circuitry (including a transmitting (and perhaps also a receiving) antenna) capable at least of initiating a call to a remote patient data center and conveying vital signs data directly from chest-worn monitor 10 thereto (and preferably capable also of conveying patient medical data directly to monitor 10, as may be needed by some applications). As shown in FIG. 8B, which represents the invention in accordance with a second embodiment, device 72 may be easily hand-carried, instead of belt- or body-worn, as it is preferably equipped with a handle.

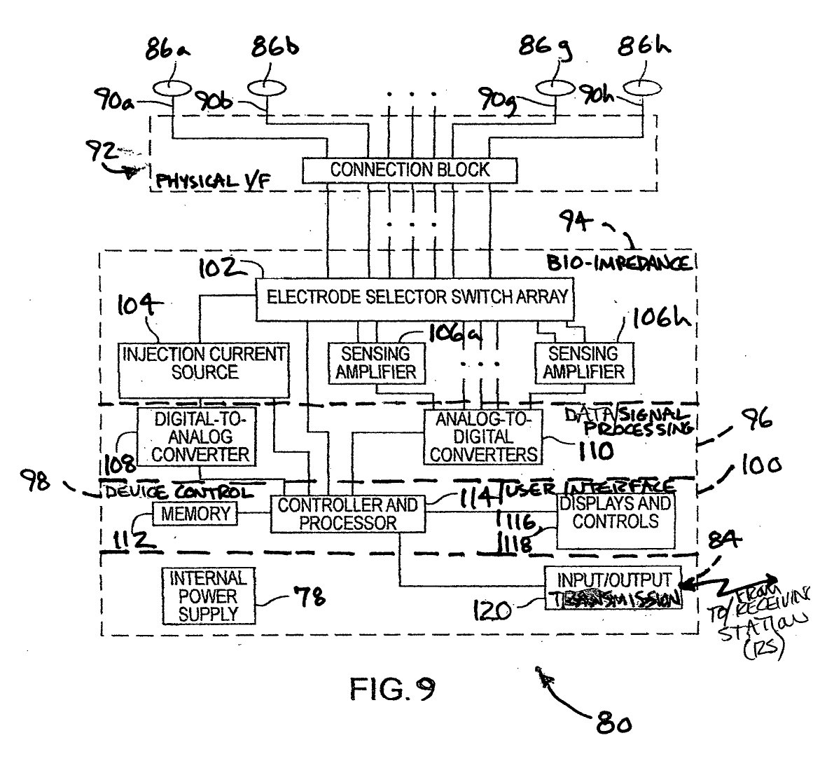

FIG. 8B illustrates that, in accordance with the invention, device 72′ may be provided operatively to connect to chest-worn device 10 in a different form. Thus, in accordance with a second embodiment of the invention, device 72′ includes a non-homeostatic body composition monitoring device 80 within a portable, hinged (openable) housing 82 that may recline on a nearby surface to employ the acoustic coupler therein or that may be hand carried by the patient when the acoustic coupler is not needed. Besides housing 82, monitoring device 80 also includes monitoring circuitry 84 (to be described below by reference to FIG. 9) and its own set of external electrodes 86a-86h (wherein, it will be understood, the electrodes may number more or fewer than eight) connectable to the patient as shown. Non-homeostatic body composition monitoring preferably is performed by way of skin bio-impedance measurements, as will be explained by reference to FIG. 9.

Those of skill will appreciate that invented device 72 including vital signs monitor 10 and invented device 72′ including body composition monitoring device 80 and two or more electrodes 86a-86h are completely external to the outer surface of the patient's skin. Thus, their use in accordance with the present invention requires no bodily invasion of any kind, whether surgical (e.g. implantation) or otherwise. Accordingly, the body composition monitor and the vital signs monitor, system and method are referred to herein as being “non-invasive.”

Device 72′ may be seen from FIG. 8B preferably also to include an I/O port 76′ corresponding generally with I/O port 76 of the first embodiment and in this embodiment operatively coupled with a telemetric means such as an acoustic coupler 88 connectable with a dial-up connection to a remote site via a conventional phone handset (not shown in FIG. 8B). Device 72′ may also be seen from FIG. 8B optionally to include I/O port 74 operatively coupled with I/O port 28 of vital signs monitor 10, thereby providing vital signs monitoring as well as body composition monitoring. Finally, device 72′ may also be seen from FIG. 8B to include an auxiliary battery 78 for supplying power to the electrical circuits of device 72′ within housing 82 thereof. Those of skill in the art will appreciate that, with hand-carry-able device 72′, no belt-worn or attire-worn container is required. Moreover, device 72′ provides full functionality to its user, in accordance with the invention, and telecommunicates data, e.g. via a modem, to a remote site via POTS if acoustic coupler 88 is used or otherwise, as by a wireless conveyance such as IR, RF, etc.

Those of skill in the art will appreciate that any suitable coupling means may be used to communicate data from device 72′ to the Receiving Station (RS). For example, a landline (POTS); a modem; a local area network (LAN); a wide area network (WAN), e.g. the Internet; a satellite link, a mobile phone or pager, or any other suitable wired or wireless means and combinations of such means may be used. Any or all such coupling and communication means are contemplated, and are within the spirit and scope of the invention.

Those of skill in the art will appreciate, electrodes 86a-86h are positioned on the patient's body in accordance with known body composition monitoring vectors similar to those vectors that are known in electrocardiography for cardiac waveform monitoring. (Vectors generally refer to current paths or measurement paths between pairs of electrodes placed on the patient's skin.) In accordance with the second embodiment of the invention, electrodes 86a-86h are configured and positioned dynamically to impress alternating current across the patient's skin and to pick-up or sense the dynamic voltages that result therefrom. Those of skill in the art will appreciate that the resulting voltages are related to the impressed currents as impedance generally in accordance with the well-known Ohm's Law represented in the formula:
E=I/R 1),
wherein E represents voltage, I represents current and R represents impedance. From formula 1) above, formula 2 is readily derived:
R=I/E 2).
Accordingly, body impedance will be understood to be the ratio of impressed current over resulting voltage. Dynamic body impedance (over time) in accordance with the invention thus is calculated using formula 2) above by a microprocessor with the variable stored current and voltage inputs as well as the variable, calculated body impedance, all stored at least temporarily in memory.

Those of skill will appreciate that as few as two electrodes are useful in body composition monitoring, in accordance with the invention. For example, two electrodes can provide a single vector of current injection and voltage response signal, three electrodes can provide as many as three vectors, four electrodes can provide as many as six vectors, etc. Thus, the eight electrodes 86a-86n shown in FIG. 8B represent a preferred embodiment of the invention that provides plural bio-impedance vectors found useful in certain cases for monitoring body composition, but the invention is not so limited to any particular number or arrangement of electrodes.

Those of skill in the art also will appreciate that the resulting dynamic body impedance, as well as other variables derived therefrom, may be stored in memory and may be conveyed, e.g. via telephone line, modem or other wired or wireless conveyance, to a remote clinic for oversight by a qualified physician. The physician may, at the remote site, record the patient's body impedance and other data, e.g. vital signs data similarly conveyed. The physician may also perform diagnostic, prescriptive and/or prognostic functions such as trend analysis and reporting. Alternatively or additionally, the resulting dynamic body impedance and other derived variables may be maintained at the patient site and the patient may interpret his or her own data and take any action that is indicated thereby. Those of skill in the art will appreciate that a patient may, over time and with a physician's assistance, learn the patient's own trends and how to analyze them and take any necessary remedial action prior to the next office visit.

Thus, a remote ambulatory patient effectively can monitor, record, review and take remedial action on his or her own vital signs and/or body composition. Alternatively, the patient can monitor, record and telecommunicate his or her own vital signs and/or body composition to a remote site for oversight by a physician or other qualified personnel, who may analyze the data and optionally diagnose and/or prescribe remedial action to be taken by the patient, caregiver or clinician. Such physician-to-patient diagnosis and/or prescription can be conveyed in the reverse direction via any suitable telecommunications conveyance, whether wired or wireless. Any suitable telecommunications conveyance or protocol is contemplated, and is within the spirit and scope of the invention.

For purposes of communicating patient data to the physician, those of skill in the art will appreciate that any suitable conveyance may be used, e.g. a phone line, the Internet (device 72′ can be equipped with an IR or USB port, for example, to a personal computer (PC)) or a wireless analog or digital network such as those used by mobile telephones. For purposes of communicating such oversight to the patient, those of skill in the art will appreciate that a display (not shown in FIG. 8A but indicated generally in FIG. 8B at block 100) may be provided for visual presentation and/or a preferably digitized memory-based recorder (see FIG. 9 at block 112) may be provided for audio presentation to the patient. Any suitable means of communicating data between an ambulatory patient and a remote physician is contemplated, and is within the spirit and scope of the invention.

At least two electrodes, and preferably more, e.g. the eight electrodes 86a-86n shown in FIG. 8B, thus convey responsive signals directly or indirectly representing the patient's skin impedance to monitoring circuitry 84 within housing 82, where the responsive signals are signal conditioned as needed, digitized and recorded in memory. For example, real-time recorded signals representing the voltages across the patient's skin can be converted to time-based digital representations of the signals and can be stored in memory. There they can be converted as described above to skin impedance data, and the patient's skin impedance data may be further processed into a form that is useful to health monitoring, i.e. they may be used to derive body fluid mass. Particular biometrics in turn can be used in trend analysis by comparing them to established general-population norms or to patient-specific norms that also are stored in memory. The memory can reside either in the monitoring device that is proximate the mobile patient or in a computer-associated mass storage device that is remote from the mobile patient, e.g. at a hospital, clinic or patient data server site. Those of skill in the art will appreciate that all such practical derivations and uses of acquired patient body composition data are contemplated and are within the spirit and scope of the invention.

Those of skill in the art will appreciate that monitoring device 72′ may be used to monitor other indicia of non-homeostatic body composition. For example, monitoring device 72′ could be configured to measure the incline of the waist of the user, which incline indicates a body conformation indicates that the wearer is overweight or obese. Or it could be configured to measure the height of the user or the user's blood pressure, cardiac output, respiratory rate, body fat, dehydration or body position/inclination (standing/sitting/reclining/reposing position) of a user. Those of skill in the art will appreciate that invented measurement device may incorporate any combination of measures of the risk of the user of monitoring device 72′ to such non-homeostatic body composition conditions as are precursors to or evidence of obesity, anorexia nervosa, bulimia or AIDS. All are within the spirit and scope of the invention.

FIG. 9 schematically illustrates a body composition ‘risk’ monitor made in accordance with an embodiment of the invention by which body fluid mass is measured via bio-impedance. The bio-impedance monitor 80 in this embodiment includes a set of electrodes 86a, 86b, . . . 86g, 86h attached to a patient via cords 90a, 90b, . . . 90g, 90h and a connection block within a physical port 92 that forms a part of housing 82. The electrodes and their cords operatively couple the patient's skin to a bio-impedance element 94. Operatively coupled to bio-impedance element 94 is a signal processing element 96, coupled in turn to device control means 99 and user interface means 100.

Bio-impedance means 94 preferably includes an electrode selector switch array 102, an injection current source 104, and plural sensing amplifiers 106a . . . 106h. Signal processing means 96 preferably includes an digital-to-analog converter (DAC) 108 operatively driving injection current source 104 and a multi-channel analog-to-digital converter (ADC) 110 operatively sampling sensing amplifiers 106a, . . . 106h. Device control means 98 preferably includes a memory 112 operatively coupled with a digital controller/processor 114, which by suitable programming controls electrode selector switch array 102, injection current source 104, DAC 108, ADC 110, memory 112 and user interface 100. User interface means 100 preferably includes one or more displays, e.g. an LCD, 116 and console controls, e.g. pushbuttons in a keypad, 118. Risk monitor 80 also may be seen to include an internal power supply, e.g. a battery, 78 and input/output (I/O) transmission means 120. Transmission means 120 provides (via I/O port 84) for the telecommunication of patient output data and physician input data to/from a remote site having a suitable sending/receiving subsystem or Receiving Station (RS), as shown. (Those of skill in the art will appreciate that examples of such a Receiving Station (RS) include an appropriately configured and programmed workstation, personal or handheld or other type of computer, Internet server, etc. Any suitable Receiving Station (RS) is contemplated, and is within the spirit and scope of the invention).

Those of skill will appreciate that fewer electrodes require fewer elements within the electrode selector switch array, fewer sensing amplifiers, fewer channels within the ADC and a simpler control algorithm within the controller and processor. Indeed, with only one pair of electrodes capable of supplying only one vector of bio-impedance data, the switch array is altogether obviated. Such alternative configurations for injecting current, monitoring voltage and deriving bio-impedance are all within the spirit and scope of the invention.

Preferably, the bio-impedance element, the user interface means the device control means, the transmission means and the power supply all are contained in a portable lightweight housing 82, as shown in FIG. 8B. The risk monitor electronics may employ wired private telephony or wireless, e.g. so-called WIFI or WiMAX, or public Internet ports to convey raw data, derived data, trend data and the like, all within the spirit and scope of the invention. It may also locally display such data for immediate monitoring and behavioral response by the patient.

The electrodes may be made of any suitable electrically conductive material, e.g. Ag—AgCl with an electrically conductive gel to couple to the patient's skin. An electrode ‘patch’ similar to that shown in FIG. 4 may be used, the patch including at least two electrically active elements or electrodes, one of which injects electrical current and the other one or more of which measures the resulting voltage. Such a patch can be used in association with other current injection and voltage measurement electrodes. Injected current is in a range between 50 μA rms and 500 μA rms, at a voltage of not greater than approximately 20V rms. The electrical current is injected at a single frequency or multiple frequencies, sequentially or concurrently. The number of discrete frequencies at which current is injected may be as many as 100 or more. The range of frequencies is from approximately 1 kHz to approximately 500 kHz. Electrical current is preferably injected with between one and twelve frequencies in the range of approximately 5 kHz to approximately 300 kHz. Multiple frequencies may be used more accurately to distinguish extracellular fluid readings. A single measurement is a single collection of readings for all frequencies and all vectors. The frequency of taking measurements ranges between approximately one hundred per second to approximately one per sixty seconds.

In brief summary, body composition or risk monitor 84 is an electronic subsystem that is capable of performing several basic tasks including repeatedly injecting current into the patient, repeatedly sensing voltages or currents resulting from the injected current, repeatedly calculating raw electrical bio-impedance data from the sensed voltages or currents and either deriving useful data therefrom and annunciating the same to the proximate patient or conveying the raw electrical bio-impedance data to a remote site for derivation therefrom of useful data and oversight by a skilled medical practitioner.

Those of skill in the art will appreciate that the memory and microprocessor are configured via programming to inject current at defined amplitudes and frequencies (via control of electrode selector switch array 102, injection current source 104 and DAC 108), to process voltage signals responsive thereto (via control of electrode selector switch array 102, sensing amplifiers 106a, . . . 106h and ADCs 110) and to store and process patient medical data, whether measured, downloaded, calculated or otherwise uploaded, e.g. weight data uploaded from a digital scale or trend data regarding body composition or individual or normative data downloaded from a remote medical patient data server. Those of skill also will appreciate that the memory and microprocessor may be used in conjunction with a display to instruct or annunciate the patient on how to use the vital signs or obesity risk monitor or what the results of various measurements and/or calculations regarding the same. Those of skill in the art will appreciate that device control means 98 common to all embodiments of the present invention include a user interface such as a display, soft or hard keyboard and cursor control means permit the user to view and/or enter data, to communicate with his or her doctor or clinic, to program settings or to input patient data and to otherwise effect user control options like automatic versus semiautomatic operation of device 72 or 72′ and/or to choose operational parameters or options. These and other alternative device control means are contemplated, and are within the spirit and scope of the invention.

FIG. 9 also illustrates the bio-impedance measurement means to be used to assess body composition in accordance with the preferred embodiment of the invention. Those skilled in the art will appreciate that bio-impedance is measured by injecting alternating current (AC) current into a patient by means of electrodes, acquiring resultant voltage characteristics, including phase angle, timing and amplitude, from the patient's body surface, e.g. skin, and calculating the resulting impedance data by correlation with and calculation from the injected current data that produced the resultant voltage characteristics, e.g. in accordance with formula 2 above.

(Those skilled in the art will also appreciate that injected current data represented of the injected current is ‘acquired’ by controller and processor 114, e.g. from electrode selector switch array 102, injection current source 104, sensing amplifiers 106a, . . . 106h, DAC 108, ADC 110 or memory 112. Thus, ‘acquired’ injected current data will be understood to represent the AC current injected via the electrodes onto the patient's skin, wherever such data resides and from whatever source such data is read or derived. It will be understood that such acquired injected current data is correlated with the resultant measured voltage data by either the local patient monitor or the remote Receiving Station (RS) to calculate the patient's electrical bio-impedance and/or body fluid composition.)

Electrical bio-impedance is a complex quantity that in a medical monitoring context represents the ratio of electrical AC current applied to and a resulting voltage measured across living tissue. The measured voltage as a function of applied frequency has amplitude and phase, or real and imaginary components and bio-impedance data would include these components, which may further include waveforms containing these elements.

Electrical bio-impedance has been used in several clinical applications, including evaluations of body composition, including both body fats and fluids, and of various hemodynamic or cardio-respiratory measurements. Several apparatuses of varying configuration and methods have been described in the background of this application which utilize bio-impedance at single or multiple frequencies for the purpose of measuring: body composition, including the distribution between extra-cellular and intracellular fluid components as well as total body water, and the distribution of fat and lean body mass; and, various hemodynamic, cardiac and respiratory measurements. The prior art further describes an apparatus similar in feature and appearance to a weight-measuring scale that further includes electrodes for use in making bio-impedance measurements to determine body fat.

In accordance with the present invention, the electrical bio-impedance values are digitized by an analog-to-digital converter (ADC) and the digital output of the ADC is analyzed by a computer or device microprocessor to detect fluid shifts within such defined volume. Fluid shifts can indicate distribution between extra-cellular fluid (ECF) and intra-cellular fluid (ICF), total body water (TBW) or fluid, changes in these components of body fluid, changes in hemodynamic and cardio-respiratory parameters including cardiac output, presence of fluid accumulations inside the body and presence, if any, of bleeding out of the circulatory system into or out of the body. In the present invention, changes in aspects of the patient's body fluid, including total body water, are of most interest, as they represent body composition characteristics that may be non-homeostatic and thus potentially indicate serious risk to the patient's overall health.

In summary of the above description of the invention, it will be understood to involve monitoring the body composition of a patient, more particularly the body fluidcontent of the patient, whereby the information regarding such body composition is communicated to a remote location by either wireless, optical, or wire-line (wired) communication. The means by which the body composition of the patient is determined is preferably through the use of bio-impedance.

The patient's body composition, e.g. information describing TBW, ICF and ECF, may be calculated by known techniques in body composition monitoring and compared and/or contrasted with previously recorded or downloaded baseline measurements to accomplish trend analysis and thus to assess patient's health risk, as indicated by non-homeostatic body composition changes. The method and device are intended for use with any patient, including humans, for whom information regarding body composition information may be useful for the purpose of monitoring at least one medical condition or disease state. More particularly, the device may be used to monitor body composition, including TBW, ICF or ECF, in patients having the condition of congestive heart failure, or the condition of kidney failure who require renal dialysis.

Thus, the invented system includes a portable, non-invasive monitoring device, generally located with a patient and including data acquisition and communication means, and a Receiving Station (RS), generally containing means of data receipt and optional processing, storage and output.

Use of body composition monitor 80 includes the following basic steps: a) attaching at least two electrically conductive electrode contacts to the patient's body, thus coupling the device to the patient for the purpose of injecting AC current and measuring resultant voltages; b) injecting AC current onto the patient's skin via the electrodes; c) acquiring voltage measurements from the patient's body responsive to and correlated with the injected current; d) optionally calculating bio-impedance measurements, including phase angle and amplitude, based upon the injected AC current and the resultant measured voltage measurements; e) optionally calculating body fluid composition from the calculated bio-impedance measurements; f) transmitting data including characteristics of at least one of the injected AC current and the measured resultant voltage measurements, the bio-impedance data and the body fluid composition data to a remote site; and g) receiving and optionally processing, storing and outputting the transmitted data at the remote site using a Receiving Station (RS).

Those of skill in the art will appreciate that, in accordance with the invention, the calculating steps can be performed at the patient monitor site or at the Receiving Station (RS) site. In the latter case, the optional steps are performed by the Receiving Station (RS) after the raw injected AC current and resultant voltage measurements data are conveyed from the patient monitor site to the Receiving Station (RS). Such post-conveyance calculations of patient bio-impedance data and optional body fluid composition data render the remote patient monitoring method equally robust but less expensive, since the patient monitor need not perform calculations that are readily performed by the Receiving Station (RS). Such bio-impedance and body fluid composition calculations, within the spirit and scope of the invention, can be performed in any suitable manner by any suitable processor at any suitable site.

Thus, the patient's bio-impedance and/or body fluid composition, more particularly the patient's body water, may also be calculated in whole or in part in the device and this information similarly transmitted to the remote site. Calculation of the bio-impedance and/or body composition information may alternatively be performed by the Receiving Station (RS). For example, only the characteristics of the injected AC current and resultant voltage measurements, preferably including the phase angle, timing and amplitude thereof, would be transmitted to a Receiving Station (RS), which would then calculate the bio-impedance and body composition. The Receiving Station (RS), for example, a computer with software and communications means to enable receipt of data from a device, may have the capability not only of receiving data, but also of performing calculations on the data, and storing and outputting the data, calculations and other information for one or more patients. The Receiving Station (RS) further includes means for enabling one-way or two-way communication with the patient by any one or more of voice communication, text messages and other visual and/or auditory signaling, as will be described further below.

Output from the Receiving Station (RS) may find use by medical professionals, including physicians, in monitoring patients having suspected or previously diagnosed medical conditions, for example, congestive heart failure, or kidney failure, or obesity, or conditions associated with body wasting. This method of monitoring would find use for both remote monitoring of such patients, for example, in their residences, or in medical facilities which otherwise have no such monitoring capability, or during medical procedures such as renal dialysis, where monitoring would occur by staff at a central Receiving Station (RS) at a distance away from the site of such procedure.

Transmission of the data may be made to a remote site that may be located within several feet of the transmitting device, for example as in a hospital or renal dialysis clinic, or the data may be transmitted to a remote site that is located dozens or hundreds or thousands of miles remote from the transmitting device, for example as in residential or institutional monitoring of congestive heart failure patients by an off-site central trans-telephonic monitoring center.

Thus, in accordance with one embodiment of the invention, the invented device corresponding generally with the invented method includes the following components: 1) a bio-impedance element capable of injecting AC current into a patient and acquiring voltages from the patient's body surface by means of electrically conductive electrodes coupling the patient to the device (see FIG. 9 at 94); 2) a communication means or means of transmitting data to a location remote from the device or patient (see FIG. 9 at 120); 3) a power supply, which may be supplied by batteries or by electrical mains (see FIG. 9 at 78); 4) a device control means, or means of controlling the device (e.g. at least one microcontroller which executes onboard instructions) (see FIG. 9 at 98); 5) a data/signal processing means, for the purpose of deriving bio-impedance information from the voltage data acquired by the bio-impedance element and enabling calculations, for example, calculating bio-impedance or body composition measurements (examples of this data processing means include an additional microprocessor or additional instructions or software placed in the device which would be executed by onboard microcontrollers or microprocessors) (see FIG. 9 at 96); and 6) controls and other user interface means to enable the patient, or a caregiver, to operate the device (see FIG. 9 at 100).

The bio-impedance or body composition information may alternatively be derived or calculated by the receiving system in addition to or instead of in the device; for example, the device might only transmit the characteristics of the injected AC current and resultant voltage measurements, including the phase, to a receiving system, which would then calculate the bio-impedance and body composition.

Device operation may be achieved with minimal or no intervention by a patient or their caregiver, following application of the electrodes, either automatically by the device control means, or remotely, by the remote receiving system interacting with the device control means. Thus, device operation may also be accomplished with only minimal interaction with controls located on the device.

The communication means may be located onboard and may comprise wireless, optical, or wire-line means for the purpose of communicating with a remote site; for example, the device may include a modem, or an onboard wireless telephone or radio, or infrared or Bluetooth® transceiver. The communication means may further include a means of receiving data or instructions from the receiving system, so that communication is two-way, for example, in addition to transmitting bio-impedance data or body composition data, the device may also receive instructions which can modify its operation or instruct the patient or caregiver. The device may also include a speakerphone capability, so that voice information may be passed from, for example, a microphone on the receiving system through to the patient using a speaker and microphone built into the device. It can easily be imagined that text sent from the receiving system could also be displayed on the device, using a display means, for example, an LCD.

The device may include an onboard means of coupling to an external transmission means for the purpose of transmitting data, instead of or in addition to an onboard communication means. For example, it may include an RJ-11 or RJ-45 or Universal Serial Bus (USB) connector or other connectors for the purpose of inserting a cable to connect the device to a telephone line, local area network, or computer or other device or network. Such coupling means may alternatively comprise optical means incorporating collimated or non-collimated light waves of any wavelength. Such wireless communication coupling means may alternatively comprise acoustic means including a speaker or emitter emitting tones having characteristics, such as frequencies, durations, or intervals, which correspond to data, such as telephone dialing tones or frequency shift key (FSK). For example, a telephone hand-piece or microphone may be placed in proximity to the speaker for purposes of conveying the tones over a telephone system. Such an acoustic device including the speaker may itself further include a microphone for the purpose of acquiring tones from the telephone handset or speaker.

The device control means enables the device to be controlled either by the patient or caregiver through the use of controls and feedback mechanisms located on the device, for example, buttons, switches, lights, light-emitting diodes (LEDs), annunciators or speakers emitting audible signals, visual displays such as a liquid crystal display, or tactile mechanisms such as vibration or other stimulation. Alternatively, the device control means may operate without human intervention by using instructions located onboard or when coupled to the receiving station by the communication means, to enable control by an operator at the receiving system or by instructions contained in the receiving station.

In addition, a data storage means, or electronic memory for the purpose of storing data or device control instructions, may be included; examples may include solid state memory, magnetic or optical media such as diskettes or compact discs, or other means, which may furthermore be removable from the device. The data contained in the data storage means may be permanently placed there, for example as with read-only solid state memory, or may be temporarily placed there, for example as with volatile random access memory.

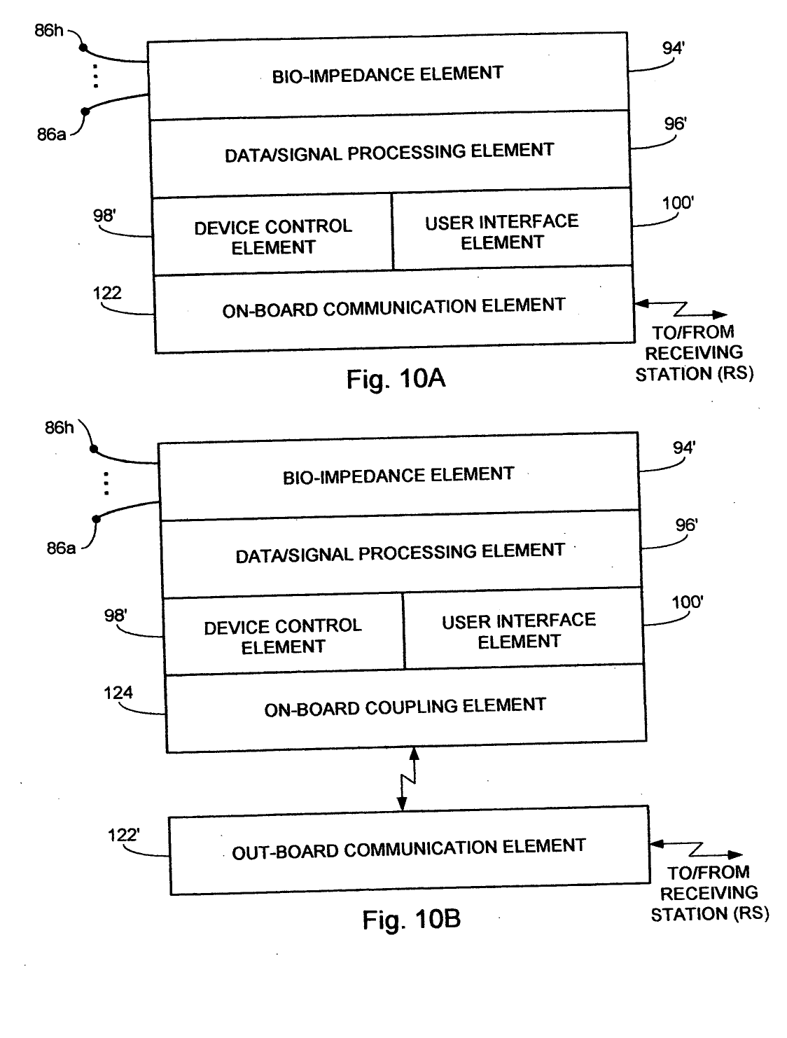

Those of skill in the art will appreciate, then, that the body composition monitor aspect of the invention may be seen to take the alternative forms illustrated schematically in FIGS. 10A and 10B. The monitor may be understood to include a set of two or more external skin electrodes 86a, . . . 86h coupled to a bio-impedance element 94′ coupled in turn to a data/signal processing element 96′ coupled in turn to a device control element 98′ and a user interface element 100′. An onboard communication element 122 may be provided ‘onboard’ the monitor, the communication element being directly coupled via a bi-directional wired or wireless data conveyance with a remote Receiving Station (RS), as shown in FIG. 10A. Alternatively, as shown in FIG. 10B, a coupling element 124 may be provided on-board to couple with an out-board communication element 122′.

Those of skill in the art will appreciate that on-board or out-board communication element 122 or 122′ can be the circuits from a landline phone, a mobile phone, a PDA, a PC or another wired or wireless conveyance. Those of skill in the art also will appreciate that the monitor shown in FIG. 10B can be combined with a landline phone, mobile phone, PDA, PC or other wired or wireless device. In this case, the landline phone, mobile phone, PDA, PC or other wired or wireless device performs the function out-board communication element 122′ and on-board coupling element 124 operatively couples the monitor circuitry thereto. Or the monitor shown in FIG. 10A can be combined with a landline phone, mobile phone, PDA, PC or other wired or wireless device. In this case, the functions of on-board communication element 122 are performed by the integral landline phone, mobile phone, PDA, PC or other wired or wireless device. Thus, any suitable integration, packaging configuration and functional/physical combination of wireless communication means with the invented body composition monitor including a bio-impedance element is contemplated as being within the spirit and scope of the invention.

In an alternative configuration, the device may further include a scale for the purpose of measuring a patient's weight, or a means of connecting to a scale, so that data about the patient's weight may be acquired, and communicated similarly to the bio-impedance or body composition data. The device may further include a means of acquiring at least one lead of ECG, acquired using the same electrodes as are used for the bio-impedance application. The device, through the data processing means, may have the additional capability of calculating cardiac output using data from the bio-impedance element of the device; alternatively, the cardiac output calculations may be made by the receiving station using, at least in part, the transmitted data, including the bio-impedance measurements. The cardiac output calculations, if supplied by the device, may be provided by a device which does not have the body fluid composition or body water information capability, and which does not have the ECG acquisition capability, but which does have the capability of transmitting data or connecting to an external transmission means. Additional biological measurements may similarly be acquired and transmitted, including respiration rate, blood pressure, and pulse oximetry measurements, as well as others which those skilled in the art can imagine.

Any suitable method of measuring or calculating body composition is within the spirit and scope of the invention. Such a calculated body composition may be compared to previously recorded or downloaded baseline measurements to determine change and rate of change, thereby to document and record trends that might indicate dehydration or other conditions of concern. It also may be compared to previously recorded or downloaded baseline measurements to determine change and rate of change, thereby to document and record trends that might indicate adverse health status.

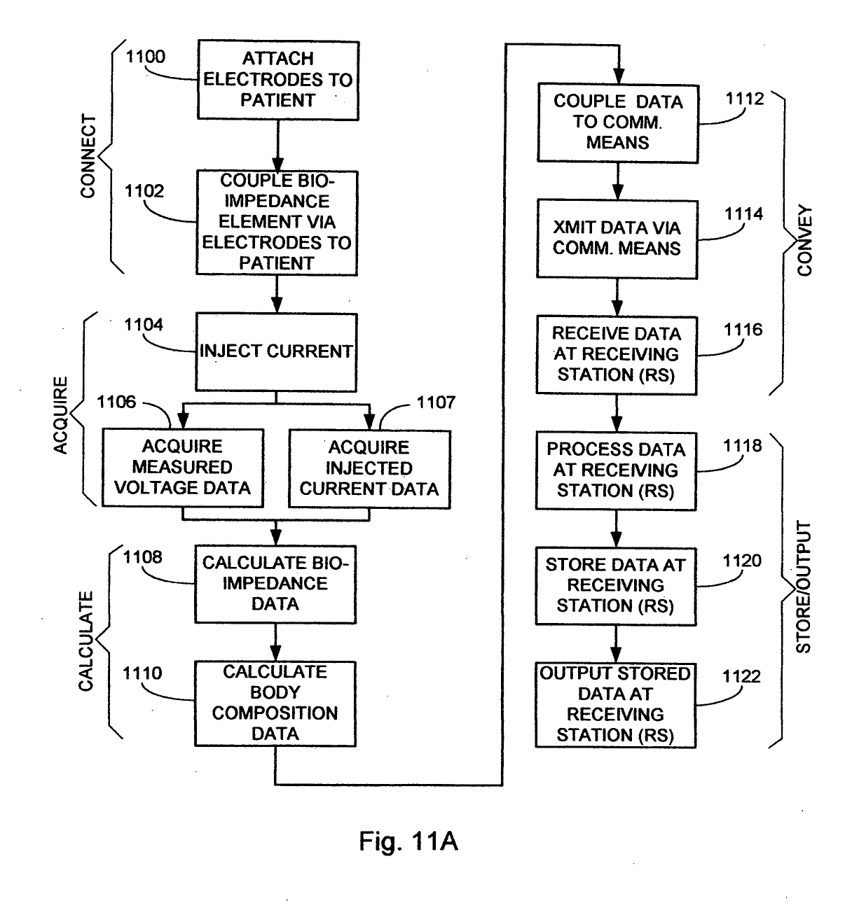

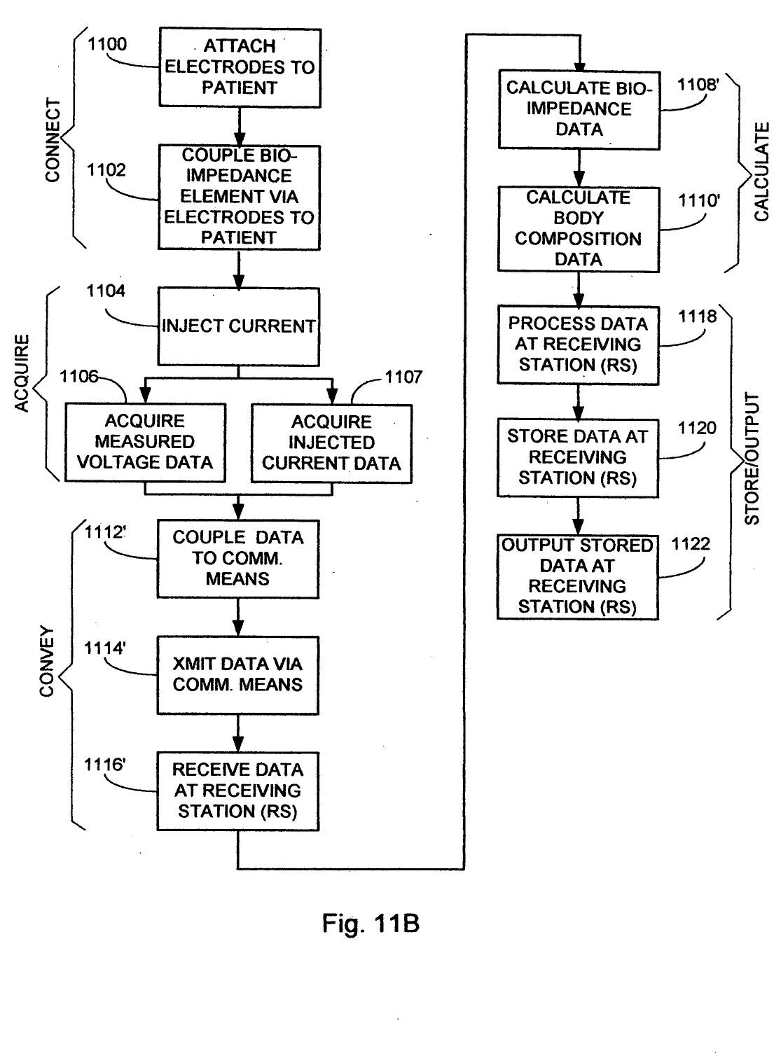

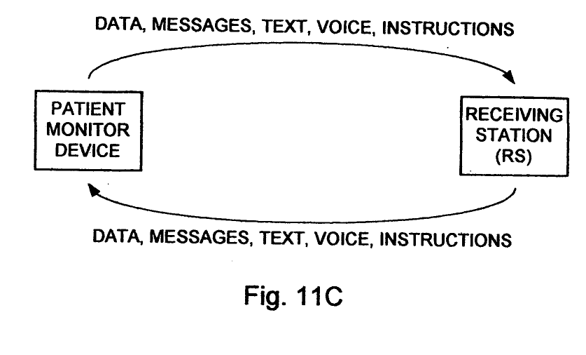

FIGS. 11A, 11B and 11C are flowcharts of the invented method in accordance with alternative aspects of the invention. It will be appreciated that FIG. 11A illustrates a method by which the patient-proximate monitor is ‘smart’ enough to derive not only bio-impedance data from the patient but also to process that bio-impedance patient data into body composition data. It will be appreciated conversely that FIG. 1I B illustrates a method by which the patient-proximate monitor injects current, collects the resultant voltages and sends data including the characteristics of the injected current, resultant voltages and phase data to a patient-remote Receiving Station (RS) where bio-impedance and optionally body composition or other data are calculated. Alternatively, the patient-proximate monitor can calculate the bio-impedance data and can send this data also to the patient-remote Receiving Station (RS). It will be appreciated that FIG. 11C illustrates the nature of the communication or conveyance between the patient monitor and Receiving Station and the variety of data, instructions and voice communication that can occur.

Referring first to FIG. 11A, the remote patient monitoring method includes a) attaching at least two electrodes that contact the patient's skin (1100); b) coupling a bio-impedance element via such electrodes to the patient to obtain at least one of AC input current data, resultant voltage data and bio-impedance data from the patient (1102); c) injecting current via such electrodes onto the patient's skin (1104); d) acquiring measured voltage data from such bio-impedance element (1106); e) acquiring injected current data corresponding to the current that was injected (1107); f) calculating bio-impedance data based upon the acquired voltage and current data (1108); and g) calculating body composition data from the calculated bio-impedance data (1110).

In accordance with a preferred embodiment of the invention, the invented method further includes h) coupling such calculated bio-impedance and body composition data to a communications means (1112); i) transmitting such calculated bio-impedance and body composition data via such communications means to a Receiving Station (RS) or other site remote from the patient (1114) j) receiving such calculated bio-impedance and body composition data at the Receiving Station (RS) or other remote site (1116); k) processing such received data at the Receiving Station (RS) or other remote site (1118); l) storing such processed data at the Receiving Station (RS) or other remote site (111820 and m) outputting such processed and stored data at the Receiving Station (RS) or other remote site (1120). It will be understood that the remote site typically would include a Receiving Station (RS), as indicated in FIGS. 8A and 8B, although sites remote from the patient monitoring site having, for example, a medical patient data server or patient medical data archive or other database and/or data collection and/or oversight equipment and/or personnel are contemplated as being within the spirit and scope of the invention.

More preferably, the method further includes acquiring, calculating, coupling and transmitting further patient data representative of at least one of body weight, electrocardiogram, respiration rate, blood pressure, cardiac output and pulse oximetry. The transmitting via such communications means preferably is wireless, wire-line or optical. Like the bio-impedance data, the body composition and other acquired and/or derived patient data preferably are stored and output at the remote site. Referring now to FIG. 11B, the alternative method is described by which the body composition data are derived from the bio-impedance data by the remote Receiving Station (RS) rather than by the patient-proximate monitor. Those of skill in the art will appreciate that, by placing the burden of converting bio-impedance data to body composition data on one or more ‘central’ Receiving Stations (RSs), relatively many invented monitors can be made at lower cost while relatively few Receiving Stations (RSs) bear only a slightly higher cost.

In FIG. 11B, identical steps are given identical reference designators while similar steps are given similar (primed) reference designators corresponding generally to those of FIG. 11A. Thus, the alternative method includes a) attaching two or more electrodes to the patient (1100); b) coupling the bio-impedance element via the electrodes to the patient (1102), c) injecting current via the bio-impedance element and the electrodes onto the patient's skin (1104); d) acquiring measured voltage data from the electrodes (1106); e) and acquiring injected current data that corresponds to the injected current (1107); f) coupling such measured voltage and injected current data to a communications means (1112′); g) transmitting such measured voltage and injected current data via such communications means to a Receiving Station (RS) or other site remote from the patient (1114′) and h) receiving such measured voltage and injected current data at the Receiving Station (RS) or other remote site (1116′).

In accordance with the alternative embodiment of the invention, the method further includes i) calculating bio-impedance data from the received measured voltage and injected current data (1108′) j) calculating body composition data from the calculated bio-impedance data (1110′); k) processing such calculated (or derived) data at the Receiving Station (RS) or other remote site (1118); l) storing such processed data at the Receiving Station (RS) or other remote site (I 120); and m) outputting such processed and stored data at the Receiving Station (RS) or other remote site (1122). In accordance with this alternative embodiment of the invention, it will be similarly understood that the remote site typically would include a Receiving Station (RS), as indicated in FIGS. 8A and 8B, although sites remote from the patient monitoring site having, for example, a medical patient data server or patient medical data archive or other database and/or data collection and/or oversight equipment and/or personnel are contemplated as being within the spirit and scope of the invention.

Those of skill in the art will appreciate that, in accordance with the alternative embodiment of the invention illustrated in FIG. 11B, the calculating steps are performed at the Receiving Station (RS) instead of at the patient monitoring device. This is made very clear by comparison of FIGS. 11A and 11B, which indicate in brackets the CONNECT, ACQUIRE, CONVEY, CALCULATE and STORE/OUTPUT phases of both methods but in two different configurations. In FIG. 11A, the phases proceed CONNECT, ACQUIRE, CALCULATE (at the patient monitor), CONVEY and STORE/OUTPUT. In contrast, in FIG. 11B, the phases proceed CONNECT, ACQUIRE, CONVEY, CALCULATE (at the Receiving Station) and STORE/OUTPUT. As described above, placing the burden of calculations such as bio-impedance and body composition on the Receiving Station instead of on the patient monitor lowers the cost of each of the relatively many patient monitors while only nominally increasing the cost of the relatively few Receiving Stations.

In accordance with either of the alternative invented methods described above by reference to FIGS. 11A and 11b, the Receiving Station (RS) also may calculate, further process, store and output other pertinent patient data transmitted by the invented monitors, whether from vital signs monitor 10 and device 72 or bio-impedance monitor 80 and device 72′. For example, non-invasive hemodynamic patient data including, for example, cardiac output or fluid content can be derived by the Receiving Station (RS) from data received from the patient monitoring site and stored and output for professional oversight by qualified medical personnel.

FIG. 11C illustrates the two-way communication that is rendered possible between the invented patient monitor device and the invented Receiving Station (RS). Such two-way communication can include one or more of data, instructions and voice. For example, Receiving Station (RS) or a health care professional operating the Receiving Station (RS) can instruct the patient via voice or text messaging where to place the bio-impedance electrodes or vital signs monitor, or can send baseline or normative data to the memory within the patient monitor device, or can ask the patient being monitored about current health, exercise, diet, chest pain or other pertinent patient inputs or observations. Similarly, the patient monitor device or the patient using the patient monitor device can inform the Receiving Station (RS) or a health care professional operating the Receiving Station (RS) via voice or text messages of any perceived health, exercise, diet, chest pain or other issues, or can send injected current data and resultant voltage measurement data (or, optionally, also calculated bio-impedance and/or body fluid composition data) to the Receiving Station (RS), or can instruct the Receiving Station to send updated baseline or normative data to the memory within the patient monitor device.

Such a message can include diagnostic or prescriptive advice to the patient from the remotely sited qualified medical personnel who is involved in oversight and review of the stored/output patient data. Or such a message can include instructions to the patient regarding use of the monitor device. Or such a message can include a simple confirmation of patient data receipt and archive. The message can be created manually by the medical professional or can be created automatically based on an intelligent response system programmed into the Receiving Station (RS) consistent with sound professional judgment. Any and all suitable communications, contents, formats, scripts and protocols are contemplated, whether one-way or two-way and whether involving data, instructions and/or voice, and are within the spirit and scope of the invention. It will be appreciated that, for the purpose of ensuring medical patient data security, patient data may be encrypted in accordance with applicable industry standards and government requirements, e.g. HIPAA regulations, before they are transmitted. Any suitable known or developed data security technique is contemplated, and is within the spirit and scope of the invention.

All such configurations of the invented vital signs monitor-whether integrated fully within a housing worn, for example, on the patient's chest or separately housed within an adherent patch-like housing and an external device worn on the patient's belt, arm, wrist, ankle or in a patient's purse, waist-pack, backpack or pocket or within a portable, grippable housing separate from the patient's body and clothing-are contemplated as being within the spirit and scope of the invention. As circuit and battery miniaturization and densification continue to increase, it is contemplated that more and more functionality may be accommodated within the confines of a conveniently portable, grippable, non-invasive, telemetric monitoring housing.

Finally, those of skill in the art will appreciate that the invented monitor, system and method described and illustrated herein may be implemented in software, firmware or hardware, or any suitable combination thereof. Preferably, the monitor, system and method are implemented in a combination of the three, for purposes of low cost and flexibility. Thus, those of skill in the art will appreciate that the monitor, system and method of the invention may be implemented by a computer or microprocessor process in which instructions are executed, the instructions being stored for execution on a computer-readable medium and being executed by any suitable instruction processor. Alternative embodiments are contemplated, however, and are within the spirit and scope of the invention.

Accordingly, while the present invention has been shown and described with reference to the foregoing preferred device and method for its use, it will be apparent to those skilled in the art that other changes in form and detail may be made therein without departing from the spirit and scope of the invention as defined in the appended claims.

1. A body composition risk monitor comprising:
a housing;
electronic circuitry within the housing, the circuitry including:
an electrode interface for operative connection with two or more external electrodes;
a signal conditioning circuit for impressing an alternating current (AC) signal across the electrode interface and for determining the impedance thereacross responsive to the impressed AC signal;
an analog-to-digital converter (ADC) for converting the determined impedance over time to a digital indicator of the user's skin's bio-impedance between two or more external electrodes; and
a digital processor for calculating defined body composition parameters from such digital indicator of bio-impedance; and
two or more external electrodes for contact with a user's skin, the electrodes being operatively connected on one end to the electronics within the housing.

2. A portable non-homeostatic body composition monitor comprising:
a housing;
a measurement device coupled with the housing to measure a defined body composition parameter;
an analysis mechanism within the housing to evaluate the measured parameter and to detect one or more changes therein sufficient to signify a trend in the defined body composition parameter that indicates a probable non-homeostatic outcome; and
a notification mechanism operatively coupled with the analysis mechanism and associated with the housing to annunciate any such detected change to a user of the monitor,
the housing, the measurement device coupled thereto, the analysis mechanism therewithin and the notification mechanism associated therewith collectively being sufficiently lightweight to be portable.

3. The monitor of claim 2, wherein the measurement device is to measure the bio-impedance of the skin of the user.

4. The monitor of claim 2, wherein the measurement device is to measure the cardiac output of the user.

5. The monitor of claim 2, wherein the measurement device is to measure the body fluid mass of the user.

6. The monitor of claim 2 which further comprises:
conveyance means operatively connected with the analysis means for wirelessly conveying to a remote site for physician oversight the results of such measurement and analysis.

7. The monitor of claim 2 which further comprises a flexible lightweight generally planar expanse having integral electrodes for adhering to the chest of the user, the expanse containing flexible electronic means of monitoring the user's cardiography and conveying the same to the risk monitor.

8. The monitor of claim 2 which further comprises:
a memory for storing data about the user; and
a mechanism for entering data in the memory.

9. The monitor of claim 8, wherein the data entering mechanism is telemetric.

10. The monitor of claim 8, wherein the data entering mechanism is manual.

11. The monitor of claim 2, wherein the measurement devices takes the form of two or more external electrodes for contact with the user's skin.

12. The monitor of claim 2 which further comprises:
a self-contained patient vital signs monitor including a generally planar expanse including a front surface and a rear surface having integral electrodes and having between the front and rear surfaces circuitry capable of sensing a vital signs signal present on the electrodes, recording the sensed signal and outplaying the recorded signal to an external device.

13. The monitor of claim 12, wherein the expanse is flexible, conforming to the shape of the patient's body and having a non-amorphous cross-sectional configuration, wherein the vital signs monitor is self-adherent to the patient's body and wherein the vital signs monitor is capable of being controlled by remote telemetry, and wherein the vital signs include ECG.

14. A system for monitoring a patient's body composition, the system including a device and a communication means, the communication means enabling communication between the device and a site remote therefrom, the device comprising:
a set of electrodes for operative coupling with a patient's skin;
a bio-impedance element operatively coupled with the set of electrodes;
a device control means for the purpose of controlling operation of the system; and
a data processor means coupled with the device control means and the bio-impedance element, and configured to obtain data from the bio-impedance element including at least one of injected AC current data, resultant voltage data, bio-impedance data, and patient body composition data.

15. The system of claim 14, wherein the device further comprises:
a housing for at least the device control means and the bio-impedance element, a display integral with the housing and a data storage means.

16. The system of claim 14, wherein the communication means is configured to communicate one or more of data, messages, text, voice and instructions between the device and the site remote therefrom.

17. The system of claim 14, wherein the communication means includes a transmitter and a receiver, the transmitter being integral with the device control means and the receiver being associable with a remote receiving station.

18. The system of claim 14, wherein the communication means enables communication of data including at least one of injected AC current and resultant voltage data, bio-impedance data, and patient body composition data.

19. The system of claim 17, wherein the transmitter is wireless.

20. The system of claim 17, wherein the transmitter is coupled to a wire-line.

21. The system of claim 17, wherein the transmitter comprises a coupling means to a communications medium located externally to the monitoring system.

22. The system of claim 14, wherein the device control means is further configured to derive from the bio-impedance element data that represents a patient's cardiac output, and wherein the communication means communicates said cardiac output data to a receiving station that is remote from the device.

23. The system of claim 14, wherein the device further comprises:
means of acquiring additional data from the patient including at least one of the patient's body weight, electrocardiogram, respiration rate, blood pressure, cardiac output and pulse oximetry.

24. The system of claim 17 which further includes at least one of a data storage means for storing data about multiple patients and a means of calculating a patient's cardiac output.

25. The system of claim 14, wherein the device further comprises:
an output means including at least one of a display and a speaker for annunciating a communication from the remote site to the patient.

26. A method of remotely monitoring a patient, the method comprising:
coupling at least two electrodes that contact the patient's skin with a bio-impedance element configured to inject electrical current onto the patient's skin and to acquire resultant voltage data from the patient's skin;
acquiring data representing such injected current and resultant voltage data from the bio-impedance element;
coupling such acquired data to a communications means; and
transmitting such acquired and coupled data via such communications means to a site remote from the patient.

27. The method of claim 26 which further comprises:
calculating bio-impedance data from the acquired injected current and resultant voltage data based upon the timing, phase angle and amplitude of the injected current and resultant voltage.

28. The method of claim 27, wherein the calculating is performed at a site proximate the patient.

29. The method of claim 27, wherein the calculating is performed at the remote site.

30. The method of claim 26 which further comprises:
receiving such transmitted data at the remote site;
processing such received data at the remote site;
storing such processed data at the remote site; and
outputting such processed data at the remote site.

31. The method of claim 27 which further comprises:
second calculating at least one of body composition data and cardiac output data from the bio-impedance data.

32. The method of claim 31, wherein the second calculating is performed at a site proximate the patient.

33. The method of claim 31, wherein the second calculating is performed at the remote site.

34. The method of claim 27 which further comprises:
acquiring, calculating, coupling and transmitting further patient data representative of at least one of body weight, electrocardiogram, respiration rate, blood pressure and pulse oximetry.

35. The method of claim 26 which further comprises:
communicating with the patient by transmitting at least one of data, a text or graphic message, a voice facsimile and an audible code from the remote site to the patient via such communications means; and
outputting the transmission to the patient utilizing at least one of a display, a speaker and other signaling or annunciating means

36. The method of claim 26, wherein said transmitting via such communications means is at least one of wireless, wire-line and optical.

37. A body composition monitoring method comprising:
collecting bio-impedance data from a patient in a non-invasive manner via a portable patient-proximate device;
conveying the collected bio-impedance data from the patient-proximate device to a remote receiving station; and
processing the received bio-impedance data at the remote receiving station to produce patient body composition data.

38. The method of claim 37, wherein the processing is performed in such manner as to produce body composition data indicative of defined hemodynamic characteristics of the patient.

39. The method of claim 38, wherein the defined hemodynamic characteristics include at least cardiac output parameters.

40. The method of claim 37 which further comprises:
conveying a message to the patient from the remote receiving station by presenting the message to the patient via at least one of a display and a speaker associated with the portable patient-proximate device.

41. A method of remotely monitoring a patient, the method comprising:
coupling at least two electrodes that contact the patient's skin with a bio-impedance element to inject electrical current and to measure resultant voltage data from the patient;
acquiring electrical current and resultant voltage data representing at least phase angle and amplitude;
second coupling such injected current data and resultant voltage data to a communication means; and
transmitting such data via such communication means to a site remote from the patient.

42. The method of claim 41 which further comprises:
receiving such transmitted data at the remote site; and
based upon such received injected current and resultant voltage data at the remote site, calculating at least one of bio-impedance data, body composition data and cardiac output data.

43. The method of claim 42 which further comprises:
coupling, acquiring, second coupling, transmitting, receiving and calculating further patient data representative of at least one of body weight, electrocardiogram, respiration rate, blood pressure and pulse oximetry.

44. The method of claim 41 which further comprises:
communicating with the patient by transmitting at least one of data, a text or graphic message, a voice facsimile and an audible code from the remote site to the patient via such communication means; and
outputting the transmission to the patient utilizing at least one of a display, a speaker and other signaling or annunciating means.
